(19) United States
(12) Patent Application Publication
(10) Pub. No.: US 2025/0058021 A1
(43) Pub. Date: Feb. 20, 2025

(54) ACOUSTICALLY-RESPONSIVE BIOINKS FOR EXTRUSION-BASED 3D-BIOPRINTING

(71) Applicant: The Regents of the University of Michigan, Ann Arbor, MI (US)

(72) Inventors: Mitra Aliabouzar, Ann Arbor, MI (US); Mario Fabiilli, Ann Arbor, MI (US)

(21) Appl. No.: 18/721,830

(22) PCT Filed: Dec. 22, 2022
(86) PCT No.: PCT/US2022/082250

§ 371 Date: Jun. 19, 2024

Related U.S. Application Data
(60) Provisional application No. 63/292,811, filed on Dec. 22, 2021.

(51) Int. Cl. A61L 27/26; A61L 27/38; A61L 27/52; A61L 27/54; B29C 64/106; B29K 105/00; B29K 105/24; B33Y 10/00; B33Y 70/00; B33Y 80/00

(52) U.S. Cl. CPC A61L 27/26; A61L 27/3834; A61L 27/52; A61L 27/54; B29C 64/106; B33Y 70/00; A61L 2300/214; A61L 2300/406; A61L 2300/414; A61L 2300/426; A61L 2300/602; A61L 2430/00; B29K 2105/0064; B29K 2105/0094; B29K 2105/24; B29K 2995/0056; B33Y 10/00; B33Y 80/00

(57) ABSTRACT

The present disclosure provides acoustically-responsive scaffold (ARS) precursor formulations comprising fibrinogen, alginate, hyaluronic acid, or a combination thereof, acoustically-responsive scaffolds comprising spatially patterned phase-shift emulsions (PSEs), and methods of using thereof (e.g., as implants, for tissue repair or regeneration and/or delivery of therapeutic agents).

### STATEMENT REGARDING FEDERALLY SPONSORED RESEARCH OR DEVELOPMENT

This invention was made with government support under HL139656 awarded by the National Institutes of Health. The government has certain rights in the invention.

### FIELD

The present disclosure provides acoustically-responsive scaffold (ARS) precursor formulations, acoustically-responsive scaffolds comprising spatially patterned phase-shift perfluorocarbon-containing emulsions, and methods of using thereof (e.g., implants, tissue regeneration, delivery of therapeutic agents).

### CROSS-REFERENCE TO RELATED APPLICATIONS

This application claims the benefit of U.S. Provisional Application No. 63/292,811, filed Dec. 22, 2021, the content of which is herein incorporated by reference in its entirety.

### BACKGROUND

Tissue micromechanics and biological function are dictated by the spatial organization and temporal dynamics of cells as well as the extracellular matrix. A major challenge in regenerative medicine is recapitulating biochemical and biophysical complexities of the native extracellular matrix within engineered constructs.

Extrusion-based bioprinting has become one of the most rapidly evolving fabrication technologies due to its precise layer-by-layer deposition of biomaterials and biological components as well as availability of multiple solidification methods. Bioprinting has enabled fabrication of multifunctional constructs with tunable mechanics and biomolecule release profiles. Several studies demonstrated that precise localization of growth factors, both spatially and temporally, in bioprinted constructs accelerated bone defect healing and angiogenesis compared to implants homogeneously loaded with the same total amount of growth factors. However, the release of biological molecules in bioprinted hydrogels is governed by mechanisms such as diffusion and degradation (either hydrolytic or enzymatic). For personalized therapy and drug delivery applications, hydrogels that respond to an externally-controlled stimulus in a user-defined, spatiotemporally-controlled manner is of great interest. Ultrasound, a clinically-used technology both in diagnostic and therapeutic applications, offers several advantages as a non-invasive stimulus including sub-millimeter precision, deep penetration within the body, and spatiotemporal characteristics. Therefore, developing bioprinted biomaterials that can be modulated with ultrasound opens new opportunities in regenerative medicine.

### SUMMARY

Disclosed herein are compositions suitable for 3D bioprinting comprising 0.5-5% (w/v) fibrinogen, 1-5% (w/v) alginate, or a combination thereof; and 0.1-2% (w/v) hyaluronic acid. The compositions are extrudable or printable into a user defined shape.

In some embodiments, the composition exhibits shear thinning behavior characterized by a decreasing viscosity with increasing shear rate. In some embodiments, the composition has a zero-shear viscosity greater than 5 Pa·s at 20° C.

In some embodiments, the compositions comprise 0.01-3% (v/v) (e.g., 0.01-1% (v/v)) of a perfluorocarbon-containing emulsion. In some embodiments, the perfluorocarbon-containing emulsion comprises perfluorocarbon droplets which vaporize from liquid droplets into gas bubbles in response to ultrasound. In some embodiments, the perfluorocarbon-containing emulsion is a double emulsion. In some embodiments, the double emulsion is a water in perfluorocarbon in water double emulsion or an oil in perfluorocarbon in water double emulsion.

In some embodiments, the perfluorocarbon-containing emulsion comprises one or more active agents. In some embodiments, the one or more active agents are conjugated to the droplet surface. In some embodiments, the one or more active agents are encapsulated within the droplet. In some embodiments, the active agent comprises a biomolecule, a therapeutic agent, a contrast agent, a detectable marker or label, or any combination thereof.

In some embodiments, the compositions comprise a plurality of cells. In some embodiments, the cells comprise progenitor cells, undifferentiated cells differentiated cells, or a combination thereof.

Also disclosed herein are acoustically-responsive scaffolds comprising a hydrogel comprising fibrin, alginate, hyaluronic acid, or a combination thereof and at least one spatially-patterned perfluorocarbon-containing emulsion. In some embodiments, the scaffolds comprise two or more spatially-patterned perfluorocarbon-containing emulsions. In some embodiments, the hydrogel comprises aligned fibrin fibers.

In some embodiments, the scaffolds further comprise a rigid hydrogel layer.

In some embodiments, the perfluorocarbon-containing emulsion comprises perfluorocarbon droplets which vaporize from liquid droplets into gas bubbles in response to ultrasound. In some embodiments, the perfluorocarbon-containing emulsion comprises one or more active agents. In some embodiments, the one or more active agents are conjugated to the droplet surface. In some embodiments, the one or more active agents are encapsulated within the droplet. In some embodiments, the active agent comprises a biomolecule, a therapeutic agent, a contrast agent, a detectable marker or label, or any combination thereof.

In some embodiments, the scaffolds comprise a plurality of cells. In some embodiments, the cells comprise progenitor cells, undifferentiated cells differentiated cells, or a combination thereof.

Further disclosed are methods for fabricating acoustically-responsive scaffolds and acoustically-responsive scaffolds made by the disclosed methods. The methods comprise: providing one or more hydrogel compositions comprising: two or more of: 0.5-5% (w/v) fibrinogen, 1-5% (w/v) alginate, and 0.1-2% (w/v) hyaluronic acid, and optionally, 0.01-3% (v/v) of a perfluorocarbon-containing emulsion, a plurality of cells, or a combination thereof; and 3D printing one or more layers of the one or more compositions to form an acoustically-responsive scaffold of defined shape, wherein the acoustically-responsive scaffold comprises at least one spatially-patterned perfluorocarbon-containing emulsion.

In some embodiments, the methods comprise providing a first hydrogel composition comprising two or more of: 0.5-5% (w/v) fibrinogen, 1-5% (w/v) alginate, and 0.1-2% (w/v) hyaluronic acid; 3D printing a first layer comprising the first hydrogel composition; providing a second hydrogel composition comprising two or more of: 0.5-5% (w/v) fibrinogen, 1-5% (w/v) alginate, and 0.1-2% (w/v) hyaluronic acid, and 0.01-3% (v/v) of a perfluorocarbon-containing emulsion; 3D printing a second layer comprising the second hydrogel composition, wherein the second layer is spatially patterned in relationship to the first layer.

In some embodiments, the perfluorocarbon-containing emulsion further comprises one or more active agents.

In some embodiments, the methods further comprise providing a rigid hydrogel substrate configured to receive the one or more layers. In some embodiments, the methods further comprise 3D printing a rigid hydrogel layer.

In some embodiments, the methods comprise crosslinking the acoustically-responsive scaffold. In some embodiments, the crosslinking comprises spraying each of the one or more layers with a crosslinking solution after 3D printing. In some embodiments, the crosslinking comprises submerging the acoustically-responsive scaffold in a crosslinking solution. In some embodiments, the crosslinking solution comprises thrombin and calcium chloride.

In some embodiments, the methods result in greater alignment of fibrin fibers compared to a conventionally polymerized acoustically-responsive scaffold. In some embodiments, the methods produce a scaffold having a significantly lower storage modulus compared to a conventionally polymerized acoustically-responsive scaffold.

Additionally disclosed are methods for using the disclosed acoustically-responsive scaffolds for wound healing or tissue repair or regeneration or administration of one or more active agents. The methods comprise implanting an acoustically-responsive scaffold disclosed herein in a desired location (e.g., tissue or organ) in a subject.

In some embodiments, the methods further comprise, exposing the scaffold to one or more ultrasound frequencies, acoustic pressure thresholds, or combinations thereof.

In some embodiments, the desired location is in a soft tissue or hard tissue.

In some embodiments, the acoustically-responsive scaffold comprises non-essential amino acids, antibiotics, cytokines, growth and morphogenic factors, or a combination thereof.

In some embodiments, the delivery of the one or more active agents is controlled spatially, temporally, or a combination thereof. In some embodiments, any of all of the one or more active agents are delivered at the same or different times as a result of exposing the scaffold to different ultrasound frequencies, acoustic pressure thresholds, or a combination thereof.

Other aspects and embodiments of the disclosure will be apparent in light of the following detailed description and accompanying figures.

### BRIEF DESCRIPTION OF THE DRAWINGS

FIGS. 1A-1C show that bioprinting enables fabrication of acoustically-responsive scaffolds (ARSs) with spatially patterned phase-shift double emulsions and high-resolution structures. FIG. 1A is an exemplary schematic illustration of bioprinting fibrin- or alginate-based ARSs. Monodispersed, micron-sized phase-shift emulsions (PSEs) with three different fluorescently-labeled dextrans were produced using a microfluidic technique: AF₄₈₈ (shown in green), AF₆₄₇ (shown in red), and AF₅₅₅ (shown in yellow). The radial distributions of flow velocity (u) and shear stress (τ) are shown in the needle with their maximum magnitudes in the center and at the wall, respectively. Scale bar: 5 μm. FIG. 1B is a schematic showing that unlike conventional polymerization techniques, bioprinting enables micropatterning of PSEs and in turn bubbles generated via acoustic droplet vaporization (ADV) within ARSs. Thus, ADV can be generated at high spatial resolutions that are otherwise not achievable based on the spatial resolution of the ultrasound used for ADV. FIG. 1C is a schematic of a bilayer construct consisting of an ARS with a lower elastic modulus (bottom layer) and a rigid hydrogel layer with a higher elastic modulus (top layer). Each layer was ˜200 μm thick. Bubbles generated by ADV in the ARS asymmetrically collapsed due to the proximity of the rigid boundary. The asymmetrical collapse, including potential of microjetting, occurred within a certain distance from the rigid boundary (d*) defined by the dimensionless standoff parameter, which is the ratio of the distance of the center of the bubble from the boundary divided by the maximum radius of the bubble. Three dimensional (3D) bioprinting enables the fabrication of ARSs with a height within the range determined by the standoff parameter.

FIGS. 2A-2F are graphs of flow behavior of the acoustically-responsive bioinks characterized by measuring the shear rate dependence of the viscosity. Measurements were conducted on the FH bioink by varying the concentration of HA (FIG. 2A), fibrinogen (FIG. 2B), and perfluorohexane (FIG. 2C) phase-shift double emulsion (C₆F₁₄, Ø=6.7±0.07 μm). The composition of FH bioink is given in Table 2. The flow behavior of the FH bioink did not exhibit significant changes over the course of 7 days (FIG. 2D). Flow behavior of three optimized acoustically-responsive bioinks (see Table 2 for the compositions of each bioink), containing 0.5% (v/v) C₆F₁₄ phase-shift double emulsions, used for bioprinting and ultrasound experiments is shown in FIG. 2E. The effect of wall slip was minimal, as seen by measuring the flow behavior of FH-C₆F₁₄ at three different measurement gaps in FIG. 2F. (N=6).

FIG. 3A is a graph of printing parameters including printing speed and extrusion pressure optimized, using conservation of mass, for three different acoustically-responsive bioinks containing 0.5% (v/v) perfluorohexane phase-shift double emulsion (C₆F₁₄, Ø=6.7±0.07 μm) (n=6). The compositions of the bioinks are given in Table 2. The volumetric flow rate from the bioprinting needle (27G, D: 200 μm, L: 6.35 mm) correlated with the extrusion pressure (FIG. 3B). Experimentally measured flow rates (n=6) were similar to values analytically determined using Eq. 6. The radial profile of shear rate (Eq. 6) in the needle was calculated based on the optimized printing parameters and rheological properties of the bioinks (FIG. 3C). The radial distribution of shear stress (Eq. 4) and the corresponding residence time (Eq. 7) in the needle for FH-C₆F₁₄ (FIG. 3D), FHA-C₆F₁₄ (FIG. 3E), AH-C₆F₁₄ (FIG. 3F) is shown.

FIGS. 4A-4E show bioprinting enabled development of acoustically-responsive scaffolds with complex geometries and precise spatial patterning of phase-shift double emulsions. FIG. 4A shows that print fidelity was evaluated by comparing the ratio of the bioprinted strand width to the original CAD model (width: 500 μm) for three developed bioinks containing 0.5% (v/v) perfluorohexane (C₆F₁₄) PSE (Ø=6.7±0.07 μm) labeled with AF₄₈₈ (shown in green). To visualize fibrin and alginate matrices, fibrinogen₆₄₇ (shown in red) and rhodamine (shown in yellow) were used, respectively. See Table 2 for compositions of each bioink. Scale bar: 100 μm. FIG. 4B is an image of an exemplary ARS (10 mm×10 mm×3 mm) bioprinted using the FHA-C₆F₁₄ bioink is shown (I) before and (II) after complete polymerization. Scale bar: 2 mm. FIG. 4C is a confocal microscopy image of a bilayer ARS containing AH-C₆F₁₄ (Ø=6.7±0.07 μm, AF₅₅₅) in the top layer and AH-C₆F₁₄ (Ø=6.7±0.07 μm, AF₄₈₈) in the bottom layer. Each layer is ˜600 μm. FIG. 4D is an image of an exemplary ARS with a more complex geometry printed with FH-C₆F₁₄ and rastered with ultrasound (US) to generate acoustic droplet vaporization. Scale bar: 2 mm. FIG. 4E is an exemplary ARS spatially patterned with three different fluorescently labeled C₆F₁₄ PSE bioprinted using the AH-C₆F₁₄ bioink. The outer (16 mm×16 mm), middle (10.6 mm×10.6 mm), and inner (5.3 mmx 5.3 mm) squares contained C₆F₁₄ PSE loaded with AF₆₄₇ (shown in red), AF₅₅₅ (shown in yellow), and AF₄₈₈ (shown in green), respectively. The zoomed in regions I and II indicate precision of bioprinting with minimal mixing at the boundaries. Scale bar: 100 μm.

FIGS. 5A-5C show bioprinting enabled micropatterning of phase-shift double emulsions (PSEs), at spatial resolutions higher than the ultrasound beam dimensions, in acoustically-responsive scaffolds (ARSs). FIG. 5A shows the width of the bubbles generated via acoustic droplet vaporization (ADV, PADV: 2.2±0.2 MPa) at the focus of the transducer (2.5 MHz, f #:0.8) measured in fibrin-based ARSs, prepared conventionally (black circles), as well as bioprinted ARSs containing 0.5% (v/v) perfluorohexane PSE (C₆F₁₄, Ø=6.7±0.07 μm) (blue circle). The width of the ultrasound beam that was suprathreshold for ADV was also measured, using a hydrophone, in free field (red squares). The focus of the transducer was rastered at a speed of 5 mm/s with a 2 mm lateral spacing between raster lines to measure the ADV-bubble width in conventional ARSs. FIG. 5B are images of an FH bioink used to bioprint an ARS with microreservoirs of C₆F₁₄ PSE (shown in green) that were 500 μm in width. The matrix also contained fibrinogen₆₄₇ (shown in red). Ultrasound (US) exposure resulted in micropatterns of ADV-generated bubbles. FIG. 5C is images of an ARS containing microreservoirs with originally designed widths of 200 μm containing two different PSEs was bioprinted according to the CAD model. Three FH bioinks were prepared to bioprint this model using three pneumatic printheads: i) FH to print the outer shell, ii) FH containing 0.05% (v/v) C₆F₁₄ (Ø=15.6±0.07 μm), and iii) FH containing 0.5% (v/v) perfluorooctane (C₈F₁₈, Φ=11.9±0.09 μm) PSEs. To enable fluorescent visualization of C₆F₁₄, and C₈F₁₈ emulsions, AF₅₅₅ (yellow), and AF₄₈₈ (green), were used, respectively. Scale bar: 200 μm.

FIGS. 6A-6F are images showing fibrin microstructure was significantly altered due to the addition of hyaluronic acid (HA) as well as bioprinting. To enable fluorescent visualization of the fibrin matrix, fibrinogen₆₄₇ (shown in red) was used. Fibrin gels, prepared conventionally, displayed a random fiber orientation without (FIG. 6A) and with (FIG. 6B) the addition of HA. Bioprinting resulted in the alignment of fibrin fibers (FIG. 6C). Hough transforms of FIG. 6A and FIG. 6C are shown in FIG. 6D and FIG. 6E, respectively. Top 4 peaks shown as black squares in FIG. 6E correspond to the detected red lines on FIG. 6F. Scale bar: 15 μm.

FIGS. 7A-7C are graphs of bulk viscoelastic moduli, including storage modulus (G′) and loss modulus (G″), of conventionally-prepared as well as bioprinted acoustically-responsive scaffolds (ARSs) characterized using a rheometer. Linear viscoelastic region was determined by conducting oscillatory strain sweep tests on ARSs containing 0.5% (v/v) perfluorohexane (C₆F₁₄, Ø=6.7±0.07) phase-shift double emulsions. ARSs with different compositions were tested: Fibrin-C₆F₁₄, FH-C₆F₁₄, FHA-C₆F₁₄, and AH-C₆F₁₄. See Table 2 for compositions of each bioink. The limit of the linear viscoelastic region, where the magnitude of G′ decreased by 5%, is represented by an arrow for each ARS (FIG. 7A). Changes in G′ and G″ in different ARSs before and after acoustic droplet vaporization (ADV) (n=4) are shown in FIGS. 7B and 7C, respectively. All ARSs exhibited a significant increase in G′ post-ADV. Statistically significant differences (p<0.05) among-ADV groups are denoted as follows: α: vs. FH-C₆F₁₄; β: vs. Fibrin-C₆F₁₄; γ: vs. FHA-C₆F₁₄.

FIGS. 8A and 8B show that bioprinting enabled micropatterning of fibroblasts in acoustically-responsive scaffolds. Normal human dermal fibroblasts were mixed in FH bioink, with a cell density of 1×10⁶ cell/mL and bioprinted in single strands using a 27-G needle and the optimized printing parameters. Cell viability in bioprinted FH gels was not significantly different than conventionally seeded FH gels (FIG. 8A). Live/dead staining was performed one hour after bioprinting and conventional polymerization (n=4). Micropatterns of fibroblasts and perfluorohexane phase-shift double emulsions (C₆F₁₄ PSE, Ø=15.6±0.07 μm), each having a width of 500 μm, were bioprinted according to the CAD model in FIG. 5B using FH bioink and three pneumatic printheads (FIG. 8B). To enable fluorescent visualization of C₆F₁₄ PSE, fibrin matrix, nuclei, and F-actin, AF₅₅₅ (shown in yellow), fibrinogen₆₄₇ (shown in red), DAPI (shown in blue), and Alexa Flour 488-labeled phalloidin (shown in green) were used, respectively. Scale bar: 200 μm.

FIGS. 9A-9D are images showing that bioprinting enabled modulation of bubble dynamics in acoustically-responsive scaffolds (ARSs). Brightfield microscopy images of bioprinted ARSs containing perfluorohexane phase shift double emulsions (Ø=15.6±0.07 μm) exposed to ultrasound (US) in the absence (FIG. 9A) and presence (FIG. 9B) of a rigid alginate layer. Interactions of the bubbles formed via acoustic droplet vaporization (ADV) with a rigid wall can induce asymmetrical collapse and/or microjetting. FIG. 9C are confocal microscopy images of the fluorescently-labeled rigid alginate wall displayed pitting and indentation likely due to the collapsed ADV-bubbles, as shown schematically in FIG. 1C. To enable fluorescent visualization of the alginate matrix, rhodamine-labeled alginate (shown in yellow) was used. FIG. 9D are surface profiles showing a maximum indentation depth of 35 μm in the rigid alginate layer. The yellow arrow shows the direction of ultrasound. Scale bar: 100 μm.

### DETAILED DESCRIPTION

Acoustically-responsive scaffolds (ARSs) comprise a hydrogel matrix doped with a perfluorocarbon-containing emulsion, termed a phase-shift emulsion (PSE). Using externally applied ultrasound, PSEs vaporize into bubbles in a process known as acoustic droplet vaporization (ADV). Bubbles are generated non-thermally within the perfluorocarbon phase of the PSE due to the rarefactional component of the acoustic wave. ARSs are typically polymerized using bulk conventional techniques that inherently limit precise, spatial patterning of the hydrogel component of the ARS as well as PSEs within the ARS. In addition, lower ultrasound frequencies, which are often used to induce ADV in ARSs due to better tissue penetration, inherently possess lower spatial resolutions compared to higher frequencies.

The present disclosure provides bioprintable ARS precursor formulations, termed acoustically-responsive bioinks, based on combinations of three natural biopolymers (fibrin, hyaluronic acid and/or alginate). These 3D-printable acoustically-responsive bioinks enabled fabrication of ARSs with precise micropatterned PSEs and in turn ADV-bubbles at high resolutions which were otherwise not achievable in conventional ARSs. The rheological properties of the bioink compositions described herein (e.g., high zero-shear viscosity and a shear thinning characteristic) are favorable for extrusion-based bioprinting offering advantages to conventional ARSs such as fabrication of ARSs with complex geometries and hydrogel compositions, precise control of the spatial distribution of multiple PSEs, and highly controlled and reproducible micropatterns in ARSs at spatial resolutions unattainable based solely on ultrasound beam dimensions, thereby facilitating better personalization of therapy. Bioprinting also yielded greater alignment of fibrin fibers in ARSs compared to conventionally polymerized ARSs.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “viscosity” refers to the resistance to flow of a material. Viscosity is reported in units of Pa·s (Pascal·second). The term “zero sheer viscosity” as used herein means the viscosity at the limit of low shear rate. In other words, the maximum plateau value attained as shear stress or shear rate is reduced. Zero-shear viscosity is effectively the viscosity of a product whilst at rest.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” and “introducing” are used interchangeably herein and refer to the placement of the scaffolds of the disclosure into a subject by a method or route which results in localization to a desired site.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Acoustically-Responsive Scaffolds

Acoustically-responsive scaffolds (ARSs) consist of a hydrogel matrix doped with a perfluorocarbon-containing emulsion, termed a phase-shift emulsion (PSE). Provided herein are acoustically-responsive scaffold (ARS) precursor compositions, or bioinks, and acoustically-responsive scaffolds comprising spatially patterned perfluorocarbon-containing phase-shift emulsions (PSEs), in some embodiments, fabricated by 3D printing with the precursor compositions.

### a) Precursor Compositions

The present disclosure provides compositions comprising, consisting of, or consisting essentially of fibrinogen, alginate, hyaluronic acid, or a combination thereof. The fibrinogen, alginate, and hyaluronic acid suitable for use in the compositions disclosed herein may be natural polymers or chemically or chemoenzymatically modified (e.g., methacrylated, thiolated, oxidated, amidated). Herein, fibrinogen, alginate, and hyaluronic acid refer to the natural polymers, chemically modified derivatives, and salt forms thereof. The fibrinogen, alginate, and hyaluronic acid are not limited to any particularly molecular weight range or distribution.

In some embodiments, the compositions comprise, consist of, or consist essentially of two or more of: 0.5-5% (w/v) fibrinogen, 1-5% (w/v) alginate, and 0.1-2% (w/v) hyaluronic acid. In some embodiments, the compositions comprise 0.5-5% (w/v) fibrinogen, 1-5% (w/v) alginate, or a combination thereof; and 0.1-2% (w/v) hyaluronic acid. In some embodiments, the compositions comprise 0.5-4% (w/v) fibrinogen and 0.2-2% (w/v) hyaluronic acid. In some embodiments, the compositions comprise 0.5-4% (w/v) fibrinogen and 1-5% (w/v) alginate. In some embodiments, the compositions comprise 1-5% (w/v) alginate and 0.1-2% (w/v) hyaluronic acid. In some embodiments, the compositions comprise 0.5-5% (w/v) fibrinogen, 1-5% (w/v) alginate, and 0.1-2% (w/v) hyaluronic acid.

The compositions may comprise 0.5-5% (w/v) (e.g., 1-5% (w/v), 2-5% (w/v), 3-5% (w/v), 4-5% (w/v), 1-4% (w/v), 2-4% (w/v), 3-4% (w/v), 1-3% (w/v), 2-3% (w/v)) fibrinogen. In some embodiments, the compositions comprise about 0.5% (w/v), about 1.0% (w/v), about 1.5% (w/v), about 2.0% (w/v), about 2.5% (w/v), about 3.0% (w/v), about 3.5% (w/v), about 4.0% (w/v), about 4.5% (w/v), or about 5.0% (w/v) fibrinogen. In select embodiments, the compositions comprise about 2.0% (w/v) fibrinogen.

The compositions may comprise 1-5% (w/v) (e.g., 1-5% (w/v), 2-5% (w/v), 3-5% (w/v), 4-5% (w/v), 1-4% (w/v), 2-4% (w/v), 3-4% (w/v)) alginate. In some embodiments, the compositions comprise about 1.0% (w/v), about 1.5% (w/v), about 2.0% (w/v), about 2.5% (w/v), about 3.0% (w/v), about 3.5% (w/v), about 4.0% (w/v), about 4.5% (w/v), or about 5.0% (w/v) alginate. In select embodiments, the compositions comprise at least about 2.0% (w/v) alginate. In select embodiments, the compositions comprise about 2.0% (w/v) to about 4.0% (w/v) alginate.

In some embodiments, the composition comprises a ratio of fibrinogen to alginate of 1:1 to 1:2 by weight.

The compositions may comprise 0.1-2% (w/v) (e.g., 0.1-1.5% (w/v), 0.1-1.0% (w/v), 0.1-0.5% % (w/v), 0.5-1.5% (w/v), 0.5-1.0% (w/v), 1-2% (w/v)) hyaluronic acid. In some embodiments, the compositions comprise at least 0.3% (w/v) hyaluronic acid. In some embodiments, the compositions comprise less than 0.75% (w/v) hyaluronic acid. In select embodiments, the compositions comprise 0.3-0.75% (w/v) hyaluronic acid.

In some embodiments, the compositions comprise about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v) or about 2.0% (w/v) hyaluronic acid.

In some embodiments, the composition comprises a ratio of hyaluronic acid to fibrinogen of 1:2 to 1:5 by weight. In select embodiments, the composition comprises a ratio of hyaluronic acid to fibrinogen of about 1:2, about 1:2.5, about 1:3, about 1:3.5, about 1:4, about 1:4.5, or about 1:5.

The compositions are configured to be extrudable or printable, for example, into a defined shape, thereby allowing fabrication of customized and complex acoustically-responsive scaffolds for biomedical applications and subject-specific therapies.

In some embodiments, the composition exhibits shear thinning behavior characterized by a decreasing viscosity with increasing shear rate. The increased shear and/or strain can be associated with extruding or printing the composition, and the viscosity of the composition can recover after extruding or printing the composition to provide a defined shape. The shear-thinning behavior allows flow through a printer nozzle at low shear rates, reducing the mechanical stress, e.g., for example stress on cells which may be in the composition. For example, the composition can have an elastic modulus (G′) higher than the loss modulus (G″) with decreased shear and/or strain on the composition and a loss modulus (G″) higher than the elastic modulus (G′) with increased shear and/or strain on the composition.

The compositions may also exhibit high, or reasonably high, zero-shear viscosity, a fast response to re-establish the high zero-shear viscosity after extrusion, and a rapid gelation to avoid deformation of the final scaffold.

The high zero-shear viscosity allows shape fidelity following printing and prevents fast sedimentation of droplets in the emulsion. The zero-shear viscosity may be significantly higher than that of conventional fibrinogen solution, which has a similar viscosity to water, of approximately 1 mPa·s. In some embodiments, the composition has a zero-shear viscosity greater than 5 Pa·s at 20° C. For example, the composition may have a zero-shear viscosity greater than 5 Pa·s, greater than 10 Pa·s, greater than 20 Pa·s, greater than 30 Pa·s, greater than 40 Pa·s, greater than 50 Pa·s, or more at 20° C.

The compositions may optionally further contain an agent or agents to assist in the polymerization of the fibrinogen, alginate, or hyaluronic acid. For example, Factor XIII can be mixed with fibrinogen to enhance polymerization.

The compositions as provided herein can optionally contain non-active excipients such as, but not limited to, water, preservative agents, buffering agents, electrolyte agents.

### b) Perfluorocarbon-Containing Emulsions

The compositions disclosed herein may comprise a perfluorocarbon (PFC)-containing emulsion (e.g., up to 3% (v/v). Higher concentrations emulsions can cause shear induced aggregations and 3D printing difficulties. The perfluorocarbon-containing emulsion comprises perfluorocarbon droplets which vaporize from liquid droplets into gas bubbles in response to ultrasound.

The compositions may comprise 0.01-3% (v/v) of a perfluorocarbon-containing emulsion. In some embodiments, the compositions comprise 0.01-2.5% (v/v), 0.01-2.0% (v/v), 0.01-1.5% (v/v), 0.01-1.0% (v/v), 0.01-0.5% (v/v), 0.01-0.25% (v/v), 0.01-0.1% (v/v), 0.01-0.05% (v/v), 0.05-3.0% (v/v), 0.05-2.5% (v/v), 0.05-2.0% (v/v), 0.05-1.5% (v/v), 0.05-1.0% (v/v), 0.05-0.5% (v/v), 0.05-0.25% (v/v), 0.05-0.1% (v/v), 0.1-3.0% (v/v), 0.1-2.5% (v/v), 0.1-2.0% (v/v), 0.1-1.5% (v/v), 0.1-1.0% (v/v), 0.1-0.5% (v/v), 0.1-0.25% (v/v), 0.25-3.0% (v/v), 0.25-2.5% (v/v), 0.25-2.0% (v/v), 0.25-1.5% (v/v), 0.25-1.0% (v/v), 0.25-0.5% (v/v), 0.5-3.0% (v/v), 0.5-2.5% (v/v), 0.5-2.0% (v/v), 0.5-1.5% (v/v), 0.5-1.0% (v/v), 1.0-3.0% (v/v), 1.0-2.5% (v/v), 1.0-2.0% (v/v), 1.0-1.5% (v/v), 1.5-3.0% (v/v), 1.5-2.5% (v/v), 1.5-2.0% (v/v), 2.0-3.0% (v/v), 2.0-2.5% (v/v), or 2.5-3.0% (v/v) of a perfluorocarbon-containing emulsion. In select embodiments, the compositions comprise 0.01-1% (v/v) (e.g., about 0.01% (v/v), about 0.025% (v/v), about 0.05% (v/v), about 0.01% (v/v), about 0.5% (v/v), or about 1% (v/v)) of a perfluorocarbon-containing emulsion

Perfluorocarbon used in emulsions suitable for ADV applications possess bulk boiling points that are lower than normal body temperature (37° C.), such as perfluoropentane (29° C. boiling point) or higher than 37° C., such as perfluorooctane (105.9° C. boiling point). Low boiling point PFCs, such as perfluoropentane, also enable the use of lower acoustic amplitudes to generate ADV and the production of stable gas bubbles in vivo. In some embodiments, the PFC emulsion comprises perfluoropropane, perfluorobutane, perfluoropentane, perfluorohexane, perfluoroheptane, perfluorooctane, or a combination thereof.

In some embodiments, the emulsion is a double emulsion. Double emulsions comprising PFC droplets are known in the art, and are described in Fabiilli et al., Pharm Res. 27(12): 2753-2765 (2010), incorporated herein by reference in its entirety. In some embodiments, the double emulsion comprises a primary (water-in-PFC) and a secondary emulsion (water-in-PFC-in-water), and is one in which aqueous droplets are suspended within a PFC droplet and have the following structure: water-in-PFC-in-water (W₁/PFC/W₂). In some embodiments, the double emulsion would be oil-in-PFC-in-water double emulsion, oil referring herein to a phase capable of solubilizing a lipophilic substances.

In some embodiments, the perfluorocarbon-containing emulsion comprises more than one population or type of PFC droplet. For example, the different populations or types of PFC droplets may comprise different perfluorocarbons, may be different types of emulsions (single vs. double), or may have different vaporization properties (e.g., vaporizing at different ultrasound frequencies and/or acoustic pressure thresholds, generating stable or transient bubbles).

In some embodiments, the perfluorocarbon-containing emulsion may further comprise a surfactant. The surfactant may stabilize the emulsion. The nature of the surfactant is largely based on the type of emulsion being stabilized. For example, a suitable surfactant for stabilizing a water-in-PFC-in-water emulsion may comprise an aqueous soluble surfactant, including but not limited to proteins, lipids, ionic copolymers, and non-ionic copolymers.

### c) Acoustically-Responsive Scaffolds

The present disclosure further provides acoustically-responsive scaffolds comprising a hydrogel composition comprising fibrin, alginate, hyaluronic acid, or a combination thereof.

In some embodiments, the acoustically-responsive scaffolds comprise a spatially-patterned perfluorocarbon-containing emulsion. The perfluorocarbon-containing emulsion comprises perfluorocarbon droplets which vaporize from liquid droplets into gas bubbles in response to ultrasound. Descriptions and embodiments of the perfluorocarbon-containing emulsion described above in relation to the disclosed compositions are applicable to the described scaffolds.

Spatially patterned, as used herein, refers to a defined pattern or patterns of one or more perfluorocarbon-containing emulsions in reference to at least one dimension of the scaffold (e.g., parallel or perpendicular to the thickness of the scaffold or in discreet domains or regions of the scaffold). Thus, spatially-patterned perfluorocarbon-containing emulsions can be targeted to a single region or multiple regions of the scaffold (e.g., at multiple depths within the scaffold or regions of the scaffold configured to interact with desired tissues or organs).

In some embodiments, the scaffold comprises more than one population or type of perfluorocarbon-containing emulsion. For example, the different populations or types of perfluorocarbon-containing emulsion may comprise different perfluorocarbons, may be different types of emulsions (single vs. double), or may have different vaporization properties (e.g., vaporizing at different ultrasound frequencies and/or acoustic pressure thresholds). The more than one perfluorocarbon-containing emulsions may have the same or different spatial patterning.

In some embodiments, the hydrogel composition of the acoustically-responsive scaffolds comprises aligned fibrin fibers. Fiber alignment may influence the mechanical properties of the hydrogel and its functional behavior (e.g., cell alignment, proliferation, and differentiation in tissue regeneration). In some embodiments, the fiber alignment is spatially-patterned, allowing tailoring of cellular responses in the acoustically-responsive scaffolds, migration of the PFC droplet and/or bubble generation, and active agent release kinetics.

The device may comprise different hydrogel layers with alternative mechanical and/or rheological properties. In some embodiments, the scaffold further comprises a rigid hydrogel layer, e.g., a hydrogel layer with a higher elastic modulus than the hydrogel comprising fibrin, alginate, hyaluronic acid, or a combination thereof. In some embodiments, the rigid hydrogel has an elastic modulus (G′) higher than the loss modulus (G″). A layer with higher elastic modulus (e.g., a rigid layer) can be used to control or direct the acoustic droplet vaporization dynamics, release kinetics, and migratory permissiveness of scaffolds. The rigid layer may comprise, consist of, or consist essentially of alginate.

### d) Active Agents

In some embodiments, the perfluorocarbon-containing emulsions of the disclosed compositions or scaffolds comprise one or more active agents. In some embodiments, a single perfluorocarbon-containing emulsion or type of PFC comprises one or more active agents. In some embodiments, the compositions or scaffolds comprise more than one type of PFC droplet or perfluorocarbon-containing emulsion, each comprising distinct active agent(s).

“Active agent” as used herein refers to any compound useful for therapeutic, prophylactic, or diagnostic purposes (e.g., any compound that is administered to a subject for the treatment, prevention, or diagnosis of a condition.). The active agent may comprise a biomolecule, a therapeutic agent, a contrast agent, a detectable marker or label, or any combination thereof.

In some embodiments, the active agent is a therapeutic agent. Generally, any therapeutic agent can be encapsulated or tethered to the perfluorocarbon-containing emulsions depending, for example, upon the condition to be treated. As used herein, the term “therapeutic agent” generally means a molecule, group of molecules, complex or substance administered to a subject for diagnostic, therapeutic, preventative medical, or veterinary purposes. Therapeutic agents encompass proteins, peptides, antigens, immunogens, vaccines, antibodies or portions thereof, antibody-like molecules, enzymes, nucleic acids, siRNA, shRNA, aptamers, small molecules, antibiotics, and any combinations thereof, including, but not limited to, topical, localized and systemic human and animal pharmaceuticals, treatments, remedies, nutraceuticals, cosmeceuticals, biologicals, and contraceptives, including preparations useful in clinical and veterinary prevention, prophylaxis, healing, wellness, therapy, surgery, cosmetics, prosthetics, and the like.

The term “therapeutic agent” also includes an agent that is capable of providing a local or systemic biological, physiological, or therapeutic effect in the biological system to which it is applied. Exemplary therapeutic agents include, but are not limited to, anti-inflammatory agents, anti-infective agents (including antibacterial, antifungal, antiviral, antiprotozoal agents), anti-allergic agents, anti-proliferative agents, anti-angiogenic agents, anti-oxidants, neuroprotective agents, hormones, anti-microbial agents, would healing agents, chemotherapeutic agents, and the like. For example, the therapeutic agent can act to control infection or inflammation, enhance cell growth and tissue regeneration, control tumor growth, act as an analgesic, promote anti-cell attachment, and enhance bone growth, among other functions. Other therapeutic agents include prodrugs, which are agents that are not biologically active when administered but, upon administration to a subject are converted to biologically active agents through metabolism or some other mechanism.

Exemplary therapeutic agents include, but are not limited to, those found in Harrison's Principles of Internal Medicine, 13th Edition, Eds. T. R. Harrison et al. McGraw-Hill N.Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference.

In some embodiments, the active agent is a biomolecule. In some embodiments, the biomolecule promotes tissue formation, destruction, and/or targets a specific disease state (e.g., growth promoters, growth factors, vitamins, minerals, enzymes, proteins, sugars, sugar alcohols). Examples include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, nucleic acids or polynucleotides (e.g., DNA or RNA encoding proteins, interfering RNA molecules), matrix metalloproteinases, tissue inhibitor of metalloproteinase enzymes, proteoglycans, glycoproteins, and glycosaminoglycans.

In some embodiments, the active agent comprises a detectable marker or label. It will be understood that a label contemplated by the disclosure includes chemiluminescent molecules, radioactive labels, dyes, fluorescent molecules (e.g., small synthetic compounds or fluorescent proteins), and phosphorescent molecules, as well as other detectable labels known in the art. The detectable marker or label may be used alone, or they may be attached to another active agent (e.g., a therapeutic agent) using methods known in the art.

In some embodiments, the perfluorocarbon-containing emulsion comprises one or more active agents conjugated to the droplet surface. In these embodiments, the emulsion may comprise a higher droplet concentration due to decreased availability for loading the active agent into the emulsion.

In some embodiments, the one or more active agents are encapsulated within the droplets of the emulsion. In some embodiments, the active agent is hydrophilic or lipophilic and a double emulsion is used to carry the agents.

### e) Cells

The compositions and scaffolds disclosed herein may further comprise a plurality of cells. In some embodiments, the cells are spatially-patterned in the scaffold.

The term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Examples of progenitor cells can include totipotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

Choice of cells will depend upon the particular downstream use of the compositions and related scaffold. The cells of the compositions and scaffolds described herein can be any cells including, for example, differentiated cells, undifferentiated cells, stem cells and/or progenitor cells with a cell lineage potential that corresponds to a tissue. The cells can be unipotent, oligopotent, multipotent, or pluripotent. In some embodiments, the cells are adult stem cells. The cells can be allogeneic or autologous. In particular embodiments, the cells include mesenchymal stem cells (MSCs). The cells can be animal cells, such as human cells. The compositions or scaffolds can contain a single cell type, or two or more different types of cells, e.g., cells of two or more different lineages.

### f) Methods of Fabrication

Provided herein are methods for fabricating an acoustically-responsive scaffold. The methods comprise: providing one or more hydrogel compositions comprising two or more of: 0.5-5% (w/v) fibrinogen, 1-5% (w/v) alginate, and 0.1-2% (w/v) hyaluronic acid, and, optionally 0.01-3% (v/v) of a perfluorocarbon-containing emulsion, a plurality of cells, or a combination thereof; and 3D printing one or more layers of the one or more compositions to form an acoustically-responsive scaffold of defined shape.

In some embodiments, the acoustically-responsive scaffolds are fabricated using one or more of the compositions as disclosed herein. Descriptions and embodiments of the perfluorocarbon-containing emulsions and hydrogel compositions described above in relation to the disclosed compositions are applicable to the described methods.

In some embodiments, the acoustically-responsive scaffold comprises at least one spatially-patterned perfluorocarbon-containing emulsion. For example, the methods may comprise providing a first hydrogel composition comprising two or more of: 0.5-5% (w/v) fibrinogen, 1-5% (w/v) alginate, and 0.1-2% (w/v) hyaluronic acid, 3D printing a first layer comprising the first hydrogel composition, providing a second hydrogel composition comprising two or more of: 0.5-5% (w/v) fibrinogen, 1-5% (w/v) alginate, and 0.1-2% (w/v) hyaluronic acid, and 0.01-3% (v/v) of a perfluorocarbon-containing emulsion, 3D printing a second layer comprising the second hydrogel composition, wherein the second layer is spatially patterned in relationship to the first layer.

In some embodiments, the methods further comprise 3D printing one or more additional layers comprising the first hydrogel composition or the second hydrogel composition. Any or all of the first hydrogel compositions or second hydrogel compositions may optionally further comprise a plurality of cells or an active agent. Thus, layering of the first hydrogen composition and second hydrogel composition facilitates spatial patterning of the perfluorocarbon-containing emulsion, and additionally, facilitates spatial patterning of the plurality of cells and active agents in complex acoustically-responsive scaffolds.

The acoustically-responsive scaffolds may comprise one or more active agents. In some embodiments, the perfluorocarbon-containing emulsion comprises one or more active agents. Thus, in some embodiments, the active agent(s) is spatially patterned as a result of the spatial patterning of the perfluorocarbon-containing emulsion. In some embodiments, the methods comprise providing and 3D printing a plurality of second layers, each comprising a hydrogel composition comprising a perfluorocarbon-containing emulsion with the same or different active agent(s).

The acoustically-responsive scaffolds may comprise a plurality of cells. For example, the methods may comprise providing and 3D printing a cellular layer comprising a hydrogel composition comprising cells. Alternatively, each hydrogel composition used for forming each layer of the acoustically-responsive scaffold may comprise cells. In some embodiments, the cells in each layer may be the same or different. Thus, the cells may be one or more layers, may be spatially patterned, or may be homogeneous throughout.

In some embodiments, the 3D printing comprises bioprinting. Herein “bioprinting” refers to three-dimensional, precise deposition of the described hydrogels utilizing methodology that is compatible with an automated, computer-aided, three-dimensional prototyping device (e.g., a 3D printer or bioprinter).

In some embodiments, the acoustically-responsive scaffold further comprises a rigid hydrogel layer. The rigid hydrogel may be prepared in advance and the hydrogel comprising fibrin, alginate, and/or hyaluronic acid may be printed on the preformed rigid layer. Alternatively, the rigid hydrogel layer may be printed prior to printing of the other layers.

In some embodiments, the methods further comprise crosslinking the acoustically-responsive scaffold. The crosslinking may provide improved mechanical properties, such as resistance to shear or tensile loading and excessive swelling. In some embodiments, the crosslinking comprises spraying each of the one or more layers with a crosslinking solution after 3D printing. In some embodiments, the crosslinking comprises submerging the acoustically-responsive scaffold in a crosslinking solution.

In some embodiments, crosslinking solution comprises a divalent cation, such as (but not limited to) a divalent metal cation selected from the group consisting of Ca²⁺, Sr²⁺, Ba²⁺, and combinations thereof. The cation is typically, although not necessarily, present as a neutral salt; for example, Ca²⁺ may be present as calcium chloride, CaCl₂). Other less preferred divalent metal due to potentially toxicity include Pb²⁺, Cu²⁺, Cd²⁺, Ni²⁺, Zn²⁺, and Mn²⁺. In some embodiments, the crosslinking agent may comprise divalent organic cations.

Fibrinogen is proteolytically cleaved and converted to fibrin monomer in the presence of a catalyst (e.g., thrombin). The fibrin monomers can then form a matrix of crosslinked fibrin, as a result of factor XIII. In some embodiments, the acoustically-responsive scaffold is crosslinked with a second agent that has thrombin and/or factor XIII for crosslinking the fibrinogen. In some embodiments, Factor XIII can be mixed with fibrinogen prior printing to enhance polymerization

In select embodiments, the crosslinking solution comprises thrombin, factor XIII, calcium chloride, or a combination thereof.

The acoustically-responsive scaffold fabricated using the methods described herein may exhibit improved mechanical properties over conventionally polymerized acoustically-responsive scaffolds. In some embodiments, the methods result in greater alignment of fibrin fibers compared to a conventionally polymerized acoustically-responsive scaffold. Fiber alignment may influence the mechanical properties of the hydrogel and its functional behavior (e.g., cell alignment, proliferation, and differentiation in tissue regeneration). In some embodiments, the methods result in a lower storage modulus compared to a conventionally polymerized acoustically-responsive scaffold.

### 3. Methods of Use

The acoustically-responsive scaffolds disclosed herein can find use in a variety of applications including implants for hard and soft tissue, tissue regeneration and repair, particularly tissues and organs with irregularly shaped wounds, precision delivery of active agents (e.g., spatial precision or temporal precision of a single agent, precise spatial or temporal delivery of multiple agents, personalization of drug therapies), localized activation of immune system, and the like.

The present disclosure provides methods for promoting wound healing or tissue repair or regeneration comprising implanting an acoustically-responsive scaffold as disclosed herein in the desired tissue or organ in the subject. For example, the scaffolds, with or without cells or growth factors, can be implanted in diseased or damaged tissues or organs to promote tissue repair.

The acoustically-responsive scaffolds may be used for wound closure systems, including vascular wound repair devices, hemostatic dressings, tissue engineering applications, such as, for example, scaffolds for tissue regeneration, ligament prosthetic devices and in products for implantation into the human body. Additionally, the scaffolds disclosed herein can be used for organ repair replacement or regeneration strategies that may benefit from the customizable scaffolds herein, including but are not limited to, spine disc, cranial tissue, dura, nerve tissue, liver, pancreas, kidney, bladder, spleen, cardiac muscle, skeletal muscle, tendons, ligaments, and breast tissues.

In some embodiments, the scaffold comprises non-essential amino acids, antibiotics, cytokines, and growth and morphogenic factors. “Growth factor” herein refers to a protein, polypeptide, or polypeptide complex which is produced by a cell and capable of affecting itself and/or various other adjacent or distant cells. Growth factors typically affect the growth and/or differentiation of certain types of cells either genetically or in response to a number of biochemical or environmental stimuli. Some, but not all, of the growth factors are hormones. Exemplary growth factors include insulin, insulin-like growth factor (IGF), nerve growth factor (NGF), vascular endothelial growth factor (VEGF), keratinocyte growth factor (KGF), fibroblast growth factor (FGF) including basic FGF (bFGF), platelet-derived growth factor (PDGF) including PDGF-AA and PDGF-AB, bone morphogenetic protein (BMP) including BMP-2 and BMP-7, hepatocyte growth factor (HGF), transforming growth factor alpha (TGF-α), transforming growth factor Beta (TGF-β) including TGFβ1 and TGFβ3, Epidermal growth factor (EGF), granulocyte-macrophage colony-stimulating factor (GM-CSF), granulocyte colony-stimulating factor (G-CSF), interleukin-6 (IL-6), and IL-8.

In some embodiments, the desired tissue for repair or regeneration is a soft tissue. Soft tissues include any tissue not hardened by an ossification or calcification process. Soft tissues include, but are not limited to, muscles, tendons, ligaments, fat, fibrous tissue, lymph and blood vessels, fasciae, and synovial membrane.

In some embodiments, the desired tissue for repair or regeneration is a hard tissue. Hard tissues are those with are mineralized or hardened by ossification or calcification, including, for example, bone, tooth enamel, dentin, and cementum.

The present disclosure further provides methods for administering one or more active agents to a subject. The methods comprise implanting an acoustically-responsive scaffold as disclosed herein in a target site in the subject, wherein the acoustically-responsive scaffold comprises one or more active agents, and exposing the scaffold to an ultrasound frequency, acoustic pressure threshold, or a combination thereof to deliver the one or more active agents to the target site. Ultrasound frequencies between about 0.5 MHz and about 50 MHz are suitable for use with the scaffolds and methods disclosed herein.

In some embodiments, the one or more active agent is sequestered within the droplets of the perfluorocarbon-containing emulsion and is released into the scaffold and target site as a result of ultrasound frequency, acoustic pressure threshold, or a combination thereof. Since ultrasound can be focused non-invasively and at a precise depth with sub-millimeter precision, the location at which droplet vaporization and administration of the agent occurs can be controlled externally with the ultrasound, or, alternatively, the spatial patterning of the perfluorocarbon emulsion acts to inherently control the location of droplet vaporization and administration of the agent from the scaffold. In some embodiments, the methods and devices described herein enable higher precision of therapy with micropatterning of droplets compared to conventional methods, e.g., at spatial resolutions higher than the ultrasound beam dimensions.

The delivery of active agents can also be controlled through the use of multiple populations of perfluorocarbon-containing emulsions or PFC droplets which vaporize at distinct ultrasound frequency and/or acoustic pressure thresholds. For example, a first active agent may be delivered after exposing the scaffold to a first ultrasound frequency and/or acoustic pressure threshold, then, following a period of time (e.g., minutes, hours, days, or weeks), a second active agent may be delivered after exposing the scaffold to a second ultrasound frequency and/or acoustic pressure threshold. The first active agent and the second active agent may be the same or different. In some embodiments, when the first and second active agents are the same, they may be in different amounts or dosages within the multiple populations of perfluorocarbon-containing emulsions or PFC droplets. Thus, the multiple populations of perfluorocarbon-containing emulsions or PFC droplets allow for spatial and temporal delivery of active agents.

### 4. Systems or Kits

Also within the scope of the present disclosure are systems or kits that include the components of the disclosed compositions or one or more of the disclosed acoustically-responsive scaffolds. For example, in some embodiments, the systems or kits include or all of: fibrinogen, or compositions thereof, alginate, or compositions thereof, hyaluronic acid, or compositions thereof, one or more perfluorocarbon-containing emulsions or components thereof, one or more active agents, and cells.

Individual member components of the systems or kits may be physically packaged together or separately. The components of the systems or kits may be provided in bulk packages (e.g., multi-use packages) or single-use packages. The systems or kits provided herein are in suitable packaging. Suitable packaging includes, but is not limited to, vials, bottles, jars, flexible packaging, and the like.

The systems or kits can also comprise instructions for using the components of the kit. The instructions are relevant materials or methodologies pertaining to the systems or kits. The materials may include any combination of the following: background information, list of components and their availability information (purchase information, etc.), brief or detailed protocols for using the compositions, troubleshooting, references, technical support, and any other related documents. Instructions can be supplied with the systems or kits or as a separate member component, either as a paper form or an electronic form which may be supplied on computer readable memory device or downloaded from an internet website, or as recorded presentation.

It is understood that the disclosed systems or kits can be employed in connection with the disclosed methods. The system or kit may further contain additional containers or devices for use with the methods disclosed herein.

### 5. Examples

### Materials and Methods

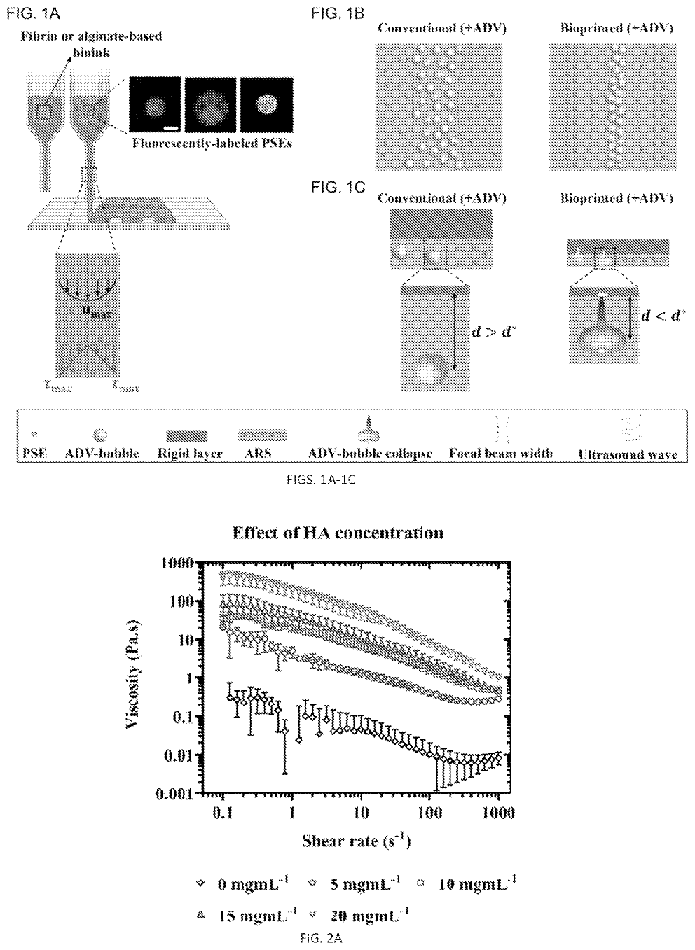

Microfluidic production of PSEs PSEs with a water (W₁)-in-perfluorocarbon-in-water (W₂) structure were manufactured using a microfluidic chip (Cat #3200146, junction: 14 μm×17 μm, Dolomite, Royston, United Kingdom) as described previously (M. Aliabouzar, et al., Ultrasound in medicine & biology 45(12) (2019) 3246-3260, incorporated herein by reference in its entirety). Perfluorohexane (C₆F₁₄, CAS #355-42-0, Strem Chemicals) or perfluorooctane (C₈F₁₈, CAS #307-34-6, Sigma-Aldrich, St. Louis, MO, USA) was used as the perfluorocarbon phase. The W₁ phase, encapsulated by a fluorosurfactant copolymer, contained fluorescently labeled dextran in phosphate buffered saline (PBS, Life Technologies). Three fluorescently-labeled dextrans were used in this study (FIG. 1A): i) Alexa Fluor 488-labeled dextran (AF₄₈₈, 10 kDa, Life Technologies, Grand Island, NY, USA), ii) Alexa Fluor 555-labeled dextran (AF₅₅₅, 10 kDa, Life Technologies), and iii) Alexa Fluor 647-labeled dextran (AF₆₄₇, 10 kDa, Life Technologies).

To produce monodispersed, micron-sized PSEs with different average diameters (Ø), the primary emulsion and W₂ phase, which was 50 mg/mL Pluronic F68 (CAS #9003-11-6, Sigma-Aldrich) in PBS, were pumped at two different flow rate combinations: i) inner and outer channels at 1 μL/min and 10 μL/min, respectively and ii) inner and outer channels at 0.5 μL/min and 2.5 μL/min, respectively. The emulsions were characterized using a Coulter Counter (Multisizer 4, Beckman Coulter, Brea, CA, USA) with a 30 μm aperture tube. Sizing characteristics of the prepared PSEs are summarized in Table 1. The type of labeled dextrans in the W₁ phase did not affect the size distribution of PSEs.

TABLE 1
Characteristics of phase-shift double emulsions used in acoustically-responsive bioinks.
Monodispersed, micron-sized emulsions with different average diameters were generated
using a microfluidic chip at different flow rate combinations (n = 3).

| | | Average | Coefficient | Number |
|---|---|---|---|---|
| | Fluorescently- | diameter | of variation | per mL |
| Perfluorocarbon | labeled dextran | (μm) | (%) | (×10⁹) |
| C₆F₁₄ | AF₄₈₈/AF₅₅₅/AF₆₄₇ | 6.7 ± 0.07 | 11.2 ± 0.6 | 3.5 ± 0.2 |
| C₆F₁₄ | AF₅₅₅ | 15.6 ± 0.07 | 2.7 ± 0.2 | 0.16 ± 0.01 |
| C₈F₁₈ | AF₄₈₈ | 11.9 ± 0.09 | 4.4 ± 0.09 | 0.45 ± 0.05 |

Preparation and polymerization of acoustically-responsive bioinks Acoustically-responsive bioinks were prepared by first adding sodium alginate powder (alginic acid sodium salt, CAS #9005-38-3, Sigma-Aldrich) to deionized (DI) water at 2-10% (w/v) and dissolving it overnight. Fibrinogen solutions were prepared by reconstituting bovine fibrinogen (Sigma-Aldrich) in PBS at 5-40 mg/mL clottable protein. Solutions were further supplemented with 0.05 U/mL aprotinin (Sigma-Aldrich), 100 U/mL penicillin, 100 μg/mL streptomycin, and 2.5 μg/mL amphotericin B (Life Technologies) and gently vortex mixed for 30 seconds. Fibrinogen and alginate solutions were then degassed in a vacuum chamber (at ˜6 kPa for 60 min, Isotemp vacuum oven, Model 282A, Fisher Scientific, Dubuque, IA, USA) at room temperature to minimize the amount of dissolved gas. To increase viscosity and induce a shear thinning characteristic in the bioinks, hyaluronic acid (HA, 1.5-1.8 MDa, CAS #9067-32-7, Sigma Aldrich) was dissolved in the prepared fibrinogen and alginate solutions under gentle stir mixing for an hour. For rheological analyses, bioinks were prepared with the following compositions: 5-40 mg/mL fibrinogen, 0.2-2% (w/v) HA, and 0.1-3% (v/v) PSE.

Acoustically-responsive bioinks containing PSEs with three finalized compositions were prepared as shown in Table 2. FHA was made by mixing separately prepared stock solutions of fibrinogen/HA and alginate at a volume ratio of 1:1. Each bioink is designated by its respective biopolymer and PSE (e.g., FH-C₆F₁₄). PSEs were incorporated into bioinks at 0.05% or 0.5% (v/v) by gentle stirring. For studies requiring fluorescence visualization of the matrix, 39 μg/mL Alexa Fluor 647-labeled fibrinogen (fibrinogen₆₄₇, F35200, Molecular Probes, Eugene, OR, USA) and rhodamine-labeled alginate (HAworks) were added into FH and AH bioinks during preparation, respectively. For the cases where asymmetrical collapse of the ADV bubbles were studied near a rigid wall, a 10% alginate bioink was prepared (FIG. 1C). Bioinks were then transferred into 3 mL syringes (Cellink) and kept at 4° C. until use. Bioinks were crosslinked with bovine thrombin (Thrombin-JMI, King Pharmaceuticals, Bristol, TN, USA) and calcium chloride (Sigma Aldrich), with the compositions used during and after printing listed in Table 2. Crosslinking solutions were sprayed after deposition of each printed layer to ensure structural integrity of the ARSs during bioprinting. After printing, ARSs were submerged in a crosslinking solution and allowed to polymerize for 30 minutes.

TABLE 2
Compositions of fibrin- and alginate-based bioinks containing
monodispersed phase-shift double emulsions. For each
bioink, crosslinking solutions with two different concentrations
were used during and after bioprinting.

| | | | | Crosslinking Solutions | |
|---|---|---|---|---|---|
| | Biopolymer | | | Thrombin (U/mL)- | |
| | | Hyaluronic | | calcium chloride | |
| | Fibrinogen | Acid | Alginate | (mg/mL) | |
| Bioink | (mg/mL) | | | During | After |
| FH | 20 | 7.5 | — | 50-0 | 20-0 |
| FHA | 20 | 4.5 | 20 | 50-50 | 20-10 |
| AH | — | 2 | 40 | 0-50 | 0-10 |

A lower concentration of the crosslinking solution was used post-printing to allow greater diffusion of crosslinker into the bioprinted construct before the outermost layers become stiff. For ultrasound experiments, bioprinted ARSs were transferred into 6-well Bioflex plates (Flexcell International, Burlington, NC, USA). For comparison, fibrin-based ARSs of similar formulation were also prepared conventionally as described previously (M. Aliabouzar, et al., Ultrasound in medicine & biology 45(12) (2019) 3246-3260, incorporated herein by reference in its entirety).

Preparation of cell-laden bioinks Normal human dermal fibroblasts (Lonza, Walkersville, MD, USA) were cultured in complete media consisting of DMEM supplemented with 10% (v/v) fetal bovine serum (FBS, Corning, Glendale, AZ, USA), 100 U/mL penicillin, and 100 μg/mL streptomycin. Media was exchanged every 2-3 days and cells were harvested below 80% confluence with trypsin-EDTA (Life Technologies). Fibroblasts were added to FH bioink at 106 cell/mL. The cell-laden bioink was loaded into 3 mL syringes and bioprinted on the same day into 6-well tissue culture plates.

Rheological characterization of bioinks Flow behaviors of bioinks were characterized using an AR-G2 rheometer (TA instruments, New Castle, DE, USA) with a parallel plate geometry (measurement head diameter: 8 mm, gap height: 1000 μm, temperature: 20° C.). To minimize slippage, adhesive-back sandpaper (600 grit, McMaster-Carr) was attached to both bounding surfaces of the rheometer. Viscosity was determined as a function of increasing shear rate from 0.1 to 1000 s⁻¹ in a stepped flow mode. The shear thinning behavior of the bioinks was characterized by fitting the linear portion of the viscosity (η) and shear rate (\dot{γ}) plots to the Oswald-de Waele power law equation:

$$η ( \dot{γ} ) = K {\dot{γ}}^{n - 1} , \qquad ( 1 )$$

where K (Pa sⁿ) is the flow consistency index and n is the dimensionless power law index. The power law index is an important factor that characterizes flow behavior. For Newtonian fluids, n=1 and K=η, however n<1 and n>1 represent non-Newtonian shear thinning and shear thickening fluids, respectively.

Bioprinting setup and process control for acoustically-responsive bioinks CAD models were created in SolidWorks (Dassault Systémes, Waltham, MA, USA) and further processed using open-source software, Slic3r (slic3r.org). The generated G-codes and STL files were sent to the 3D bioprinter (Bio X, Cellink, USA). All samples were printed using 3 mL pneumatic printheads at room temperature. A 27-gauge needle, with an internal diameter (D) of 200 μm and length (L) of 6.35 mm, was used for all prints. Experimental as well as theoretical studies were performed to understand the interrelations between printing parameters (e.g., extrusion pressure, and printing speed), needle geometry (e.g., D and L), rheological properties of the bioink (e.g., n and K from Eq. 1), and the process-induced mechanical stresses during extrusion (e.g., shear and extensional stresses) (FIG. 1A).

Experimental and theoretical flow rate studies The optimal printing speed was determined by assuming that printing speed equals extrusion velocity. The printing speed (vₚ) at a given volumetric flow rate (Q) was determined using conservation of mass. Assuming negligible spreading (e.g., a cylindrical geometry), vₚ was calculated as:

$${v}_{p} = \frac{4 Q}{π {D}^{2}} . \qquad ( 2 )$$

Q was derived by weighing the mass of bioink, using a digital scale (Mettler-Toledo, USA), dispensed through the needle for 30 s for varying extrusion pressures (3-25 kPa), and then dividing by the dispensing time. The density of the bioink was assumed to be 1 g/mL. According to Eq. 2, at a critical value of vₚ, the diameter of the printed strand will equal the diameter of the needle. For v<vₚ, the layer height will be thicker than D, while for v>vₚ, thinner layers are printed. Based on these findings, optimal printing parameters including the extrusion pressure and printing speed were obtained for each bioink.

The experimentally measured volumetric flow rates were then compared to an analytical model for flow rate of non-Newtonian fluids using a modified Hagen-Poiseuille equation:

$$Q = {π ( \frac{D}{2} )}^{3} {( \frac{D Δ p}{4 KL} )}^{\frac{1}{n}} ( \frac{n}{n + 1} ) ( 1 - \frac{2 n}{2 n + 1} + \frac{2 {n}^{2}}{( 2 n + 1 ) ( 3 n + 1 )} ) , \qquad ( 3 )$$

where Δp is the extrusion pressure.

Radial distribution of shear stress, shear rate, and residence time in the needle PSEs, which are dispersed within the bioink, experience several types of mechanical forces during the bioprinting process, including shear forces due to steady shear flow, extensional forces, and pressure drop due to the sudden geometrical transition in the flow from a syringe to a needle. Based on the principle of force balance, the bioprinting-induced shear stress (τ) distributed radially in the needle can be written as:

$$τ ( r ) = \frac{r Δ p}{2 L} , \qquad ( 4 )$$

where r is the radial position (0≤r≤D/2) in the needle. Radial flow velocity profile (u(r)) of a non-Newtonian fluid in a cylindrical needle as a function of rheological properties as well as printing parameters can be obtained using modified Hagen-Poiseuille equation:

$$u ( r ) = \frac{n}{n + 1} {( \frac{Δ p}{2 LK} )}^{1 / n} ( {( \frac{D}{2} )}^{n + 1 / n} - {r}^{n + 1 / n} ) . \qquad ( 5 )$$

Bioprinting-induced shear rate (\dot{γ}_{bp}) and the corresponding residence time (tᵣₑₛ) inside the needle can be calculated from Eq. 5 as follows:

$${\dot{γ}}_{bp} = \frac{du ( r )}{dr} = {( \frac{r Δ p}{2 LK} )}^{1 / n} , \qquad ( 6 )$$

$${t}_{res} ( r ) = \frac{L}{u ( r )} . \qquad ( 7 )$$

For the above calculations, several assumptions were made: i) the bioink is incompressible, ii) no slip occurs between PSEs and the wall of the needle, and iii) the pressure at the exit of the needle equals the ambient pressure. Note that the generalized Reynolds number for a power-law fluid through a cylindrical pipe for the varying printing conditions used here was <0.03, indicating a uniform laminar flow condition inside the bioprinting needle. Based on the calculated Reynolds number and the diameter of the needle, the flow became fully developed within micrometers from the needle entrance.

Shear-induced droplet deformation Droplet deformation and breakup under shear can be determined by the dimensionless capillary number representing the ratio of viscous to interfacial tension forces defined as:

$$Ca = \frac{η \dot{γ} ϕ}{2 σ} , \qquad ( 8 )$$

where η, \dot{γ}, ϕ, and σ represent the viscosity of the bioink (approximated by the value of K, e.g., flow consistency index), bioprinting-induced shear rate (obtained from Eq. 6), diameter of a PSE (˜12 μm), and the interfacial tension between PSE (Pluronic copolymers: 42 mN/m (N. Y. Rapoport, et al., Bubble Sci Eng Technol 1(1-2) (2009) 31-39, incorporated herein by reference in its entirety)) and the surrounding bioink, respectively.

Assessment of print fidelity Printing fidelity was defined as the ratio of the width of the bioprinted single-layer strands with different bioinks, containing 0.5% (v/v) C₆F₁₄ PSE (Ø=6.7±0.07 μm), to the strand width in the original CAD model (strand width: 500 μm).

Ultrasound setup and parameters All ultrasound experiments were carried out in a water tank (30 cm×60 cm×30 cm) filled with degassed, DI water at 37° C. A calibrated, focused transducer (H-108, f-number=0.83, radius of curvature=50 mm, Sonic Concepts Inc., Bothell, WA, USA) was used to induce ADV within the ARSs. Pulsed waveforms (2.5 MHz, pulse duration: 5.4 μs; pulse repetition frequency: 100 Hz) were generated by a function generator (33500B, Agilent Technologies, Santa Clara, CA, USA), amplified by a gated radiofrequency amplifier (GA-2500A Ritec Inc., Warwick, RI, USA), and monitored in real-time on an oscilloscope (HD04034, Teledyne LeCroy, Chestnut Ridge, NY, USA). The transducer was calibrated in free field at the focus using an in-house fiber optic hydrophone (sensitivity: 16.6 mV/MPa) with a fiber diameter of 105 μm. The acoustic pressure distribution at the focus of the transducer was characterized, using the hydrophone, to measure the focal width that was suprathreshold for ADV.

The transducer was connected to a three-axis positioning system controlled by MATLAB (The MathWorks, Natick, MA, USA) and localized axially with respect to the ARSs using a pulse echo technique described previously (M. Aliabouzar, et al., Ultrasonics sonochemistry 66 (2020) 105109). During ultrasound exposure, the axial focus of the transducer was positioned at mid-height in the ARSs, and then rastered at a speed of 5 mm/s with a 0.5 mm lateral spacing between raster lines. A single plane of exposure was deemed sufficient due to the axial length of the beam at full width half maximum at the focus. Ultrasound experiments were conducted at 6 MPa peak rarefactional pressure, which was suprathreshold for both ADV (2.2±0.2 MPa) and inertial cavitation (3.9±0.2 MPa) at 2.5 MHz for ARSs containing C₆F₁₄ PSE. ARSs not exposed to ultrasound served as controls.

Rheological characterization of the ARSs Dynamic rheological properties of ARSs (height: ˜1.2 mm, diameter: ˜8 mm) were measured using the AR-G2 rheometer. 3D bioprinted ARSs of different compositions contained 0.5% (v/v) C₆F₁₄ PSE (Ø=6.7±0.07 μm). For comparison, fibrin ARSs were also prepared conventionally with identical fibrinogen, thrombin, and C₆F₁₄ PSE concentrations. ARSs were subjected to sinusoidal oscillatory strains and the corresponding stress values were recorded. The rheometer stage was maintained at 37° C. Samples were indented to reach a normal force of 0.07 N (indentation: ˜200 μm) and allowed to equilibrate for 2 minutes. Oscillatory strain sweep (0.05-50%) was carried out at frequency of 1 Hz to determine the linear viscoelastic region (LVR). Within the LVR, the viscoelastic moduli, including the storage modulus (G′) as well as loss modulus (G″), are independent of applied strain or stress. The limit of LVR was represented as the critical strain where ′G reduced by 5%. Samples were then subjected to oscillatory shear at 1 Hz with a strain of 1% (based on the LVR) to determine G′ and G″.

Optical imaging and analyses Bioprinted ARSs were imaged with an epifluorescent microscope (Eclipse TiE, Nikon, Melville, NY, USA) and acquisition software (MetaMorph, Molecular Devices, San Jose, CA, USA). MATLAB and ImageJ (National Institutes of Health, Bethesda, MD, USA) were used for further analysis. Confocal images of ARSs were acquired in a cell chamber (Attofluor, A7816, Thermo Fisher Scientific, Waltham, MA, USA) using a laser scanning confocal microscope (LSM800, Zeiss, Pleasanton, CA, USA) and ZEN lite software (Zeiss). Selected confocal images were converted to binary edge maps and transformed in Hough space to determine bioprinting-induced fiber alignment in MATLAB.

Evaluation of cell viability and morphology in conventional and bioprinted gels To assess viability, FH constructs containing fibroblasts were stained one hour after printing with 5 μM calcein AM (“Live” stain, Invitrogen) and 15 μM propidium iodide (“Dead” stain, Invitrogen). To assess cell morphology, FH constructs containing fibroblasts were cultured four days, fixed in aqueous buffered zinc formalin (Z-Fix, Anatech, Battle Creek, MI, USA), and permeabilized with 0.1% (v/v) Triton-X100 (Sigma-Aldrich) in PBS. After triplicate washings, constructs were incubated in a blocking solution containing 0.1% (v/) Tween 20 (Sigma-Aldrich), 1% (w/v) bovine serum albumin, 10% (v/v) goat serum (Life Technologies), and 0.3 M glycine (Sigma-Aldrich) in PBS. Constructs were stained overnight at 4° C. with Alex Fluor 488-labeled phalloidin (1:400 dilution A12379, Molecular Probes). The next day, constructs were washed in triplicate and stained overnight with 1 μg/mL 4,6′-diamidino-2-phenylindole (DAPI, Thermo Fisher Scientific, Waltham, MA, USA) in PBS. After triplicate washing, constructs were imaged.

Statistical analyses Statistical analyses were performed using GraphPad Prism software (GraphPad Software, Inc., La Jolla, CA, USA). Experimental data are expressed as the mean±standard deviation. The number of independent replicates is listed in the caption for each figure. Significant differences between groups were determined using a two-tailed t-test or a one-way ANOVA followed by Tukey's multiple comparisons test. A significance level of 0.05 was used.

### Example 1

### Rheological Properties and Printability Assessment of Acoustically-Responsive Bioinks

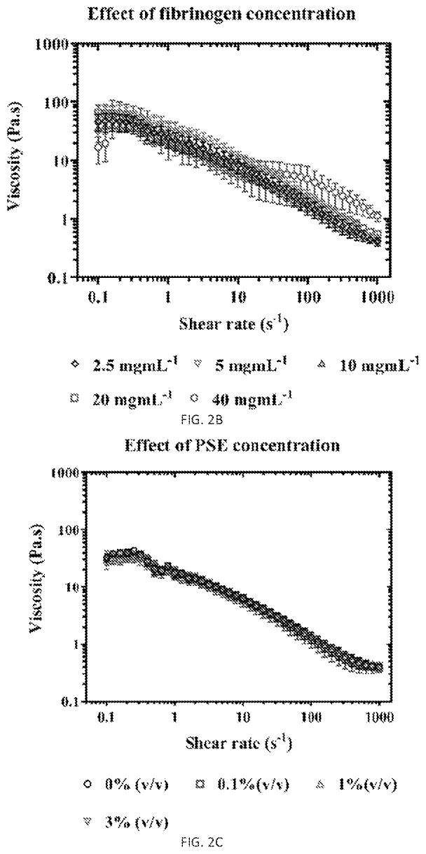

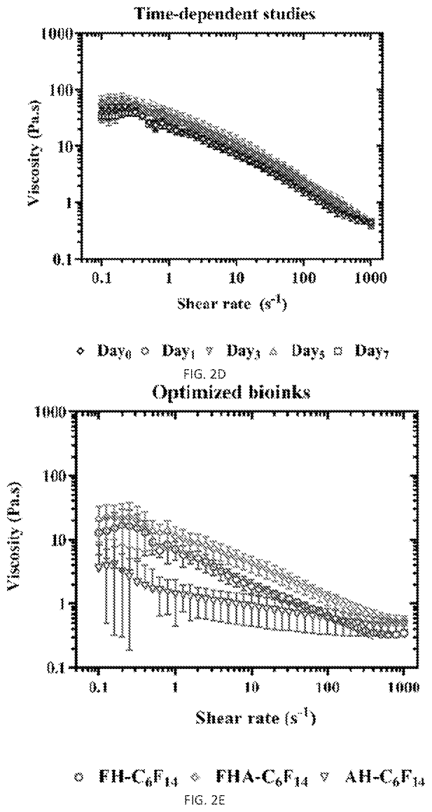

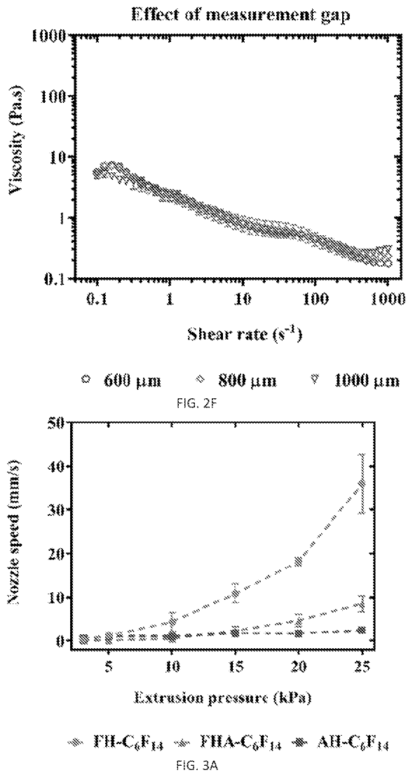

The rheological behavior of a bioink must fulfill a number of key requirements for extrusion-based bioprinting. Bioinks with shear-thinning characteristics where apparent viscosity decreases with induced shear rate are ideal due to reduced pressures required for extrusion. Fibrinogen solution is not suitable for extrusion-based bioprinting due to its significantly low viscosity, which is comparable to water, and Newtonian behavior (FIG. 2A, black diamonds). To formulate fibrin-based bioinks with favorable rheological properties and better printability, fibrinogen was blended with HA or HA/alginate. Addition of HA to fibrinogen increased the viscosity of the FH bioinks and induced non-Newtonian, shear thinning behavior characterized by a decrease in viscosity with increasing shear rate (FIG. 2A). A higher HA concentration increased viscosity at a given shear rate. However, higher concentrations of HA significantly increased fibrin polymerization time. HA at 20 mg/mL inhibited fibrin polymerization despite incubation for 8 hours. This observation was consistent with a prior study that showed inhibition of fibrin polymerization correlated with the concentration and molecular weight of HA (N. Storozhylova, et al., Regenerative Engineering and Translational Medicine 6(2) (2020) 201-216, incorporated herein by reference in its entirety). Therefore, HA concentrations of 7.5 mg/mL and 4.5 mg/mL were chosen for FH and FHA bioinks, respectively, to maintain high viscosity and shear-thinning properties as well as fast fibrin polymerization.

Although stiffness of fibrin-based gels correlated with fibrinogen concentration, fibrinogen concentration did not significantly impact the viscosity and shear thinning behavior of FH bioinks (FIG. 2B). FIG. 2B highlights that the rheological behavior of FH bioink is largely dictated by the HA component. Since suspension of particles at higher concentrations might impact the fluid flow behavior (e.g., shear-induced aggregation), similar rheological measurements were conducted on FH bioinks containing various volume fractions of C₆F₁₄ PSE (Ø=6.7±0.07 μm). As shown in FIG. 2C, inclusion of C₆F₁₄ PSE (up to 3% (v/v)) did not change the rheological behavior of the bioink. The rheological properties of the FH bioinks did not significantly change over time, indicating bioink stability for up to 7 days (FIG. 2D).

Flow behaviors of the three optimized acoustically-responsive bioinks used for bioprinting and ADV experiments (FIG. 2E) demonstrated shear thinning characteristics and high zero-shear viscosities, which are critical for extrusion-based bioprinting. Using Stoke's law, the maximum terminal velocity of the emulsions in these bioinks was 0.01 μm/s as a result of high zero-shear viscosity; therefore, minimal settling of PSEs was expected during bioprinting. By fitting viscosity-shear rate profiles in FIG. 2E to the power law regression (Eq. 1), power law variables (e.g., K and n) were determined and summarized in Table 3. The correlation coefficient (R²) for all fittings was greater than 0.91. Note that smaller values of n correspond to a greater degree of shear-thinning. Table 3 indicates that FH-C₆F₁₄ bioink had the highest degree of shear thinning compared to the other two bioinks. The possibility of slip was investigated by conducting flow measurements at three different gaps as displayed in FIG. 2F. No significant change in flow behavior and the obtained power law constants were measured, indicating the minimal effect of wall-slip.

TABLE 3
Flow consistency (K) and power law index (n) were obtained
by fitting flow behavior of acoustically-responsive bioinks
containing 0.5% (v/v) perfluorohexane (C6F14) phase-shift
double emulsions (FIG. 2E) to a power law model (Eq. 1).

| Bioink | n | K (Pa sⁿ) |
|---|---|---|
| FH-C₆F₁₄ | 0.46 ± 0.04 | 6.9 ± 0.8 |
| FHA-C₆F₁₄ | 0.55 ± 0.04 | 11.7 ± 2.3 |
| AH-C₆F₁₄ | 0.78 ± 0.01 | 1.5 ± 0.4 |

### Example 2

### Evaluation of Bioprinting Process Induced Parameters

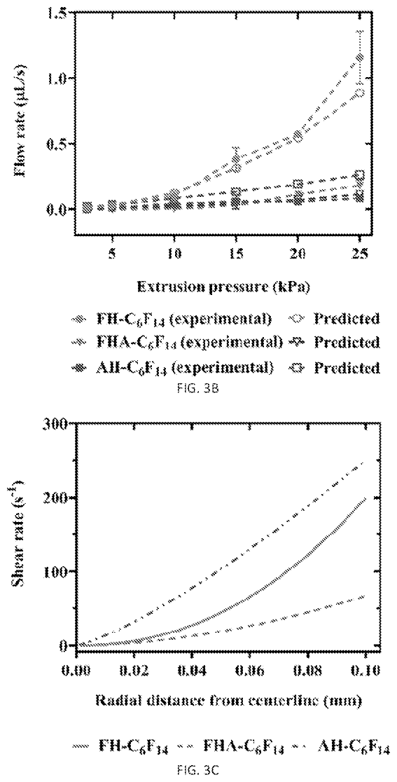

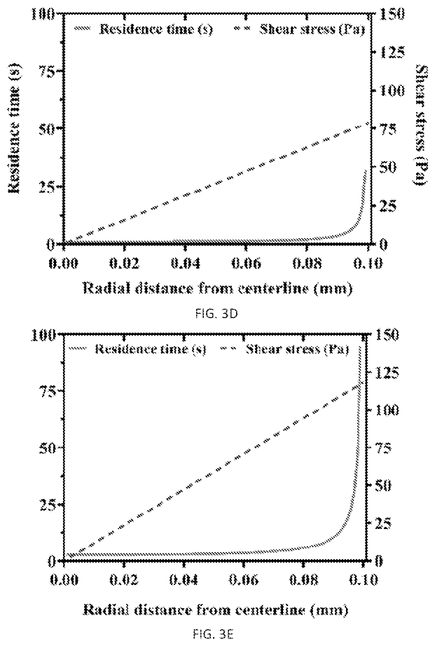

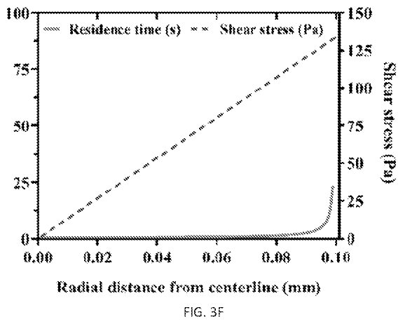

Volumetric flow rates of the bioinks were measured to optimize printing parameters including extrusion pressure and printing speed (Eq. 2). Printing speed (FIG. 3A) and volumetric flow rate (FIG. 3B) correlated directly with the extrusion pressure as well as the shear thinning degree of the bioink. At higher extrusion pressures, FH-C₆F₁₄ bioink underwent significant shear thinning, resulting in higher flow rates and consequently faster printing speeds. Extrusion pressures of 10 kPa (v=5 mm/s), 15 kPa (v=4 mm/s), and 17 kPa (v=3 mm/s) were maintained throughout the experiments for FH, FHA, and AH bioinks containing PSEs, respectively.

The radial distribution of shear rate in the needle significantly depended on the degree of shear thinning of the bioink (FIG. 3C). For all bioinks, shear rate increased toward the needle wall. This increase was almost linear for the weakly shear thinning bioink (e.g., AH-C₆F₁₄, n ˜0.78) and more non-linear adjacent to the wall for a highly shear thinning bioink (e.g., FH-C₆F₁₄, n ˜0.46). Plug flow behavior is expected for shear thinning flow, where bioinks experience a lower shear rate in a wide region in the center and a narrow zone of higher shear adjacent to the wall. The shear stress (Eq. 4) depends on the needle dimensions, which was the same for all the prints herein, as well as the extrusion pressure, which was 10 kPa, 15 kPa, and 17 kPa for FH, FHA, and AH bioinks, respectively. As can be seen in FIGS. 3D-3F, shear stress increased linearly for all bioinks to its maximum value at the needle wall and was the lowest for FH bioink.

Ca number, calculated based on the resulting shear rate and viscosity distributions, was less than 0.01 for all bioinks for the printing conditions used here. At large Ca numbers, viscous forces dominate, resulting in shape deformation from spherical to ellipsoidal and eventual breakup into smaller droplets. However, at lower Ca, surface forces dominate and maintain the droplet shape. The viscosity ratio between the dispersed phase (e.g., PSE) and continuous phase (e.g., the bioink) was <0.3. Significant droplet deformation and breakup was reported at Ca>0.35. Note that droplet deformation also increases with increasing confinement at a given Ca. Here, the ratio of the diameter of PSE to the diameter of the needle was 0.05, therefore confinement-induced deformation was negligible.

The abrupt change in the cross-sectional diameter as the bioink transitioned from the syringe to the needle (D_{syringe}/D=21), resulted in a ˜441-fold increase in the linear fluid velocity. The maximum pressure drop induced at this contraction region, using the steady flow energy equation, was ˜500 Pa at bioprinting conditions used herein. Mechanical disruption of emulsions caused by this pressure drop should be minimal. Note that, using a capillary rheometer, a pressure drop of 800 Pa was measured for a 2% (w/v) alginate ink through a needle with the same diameter (e.g., 200 μm) but at a higher extrusion pressure of 50 kPa.

### Example 3

### Micropatterning ADV

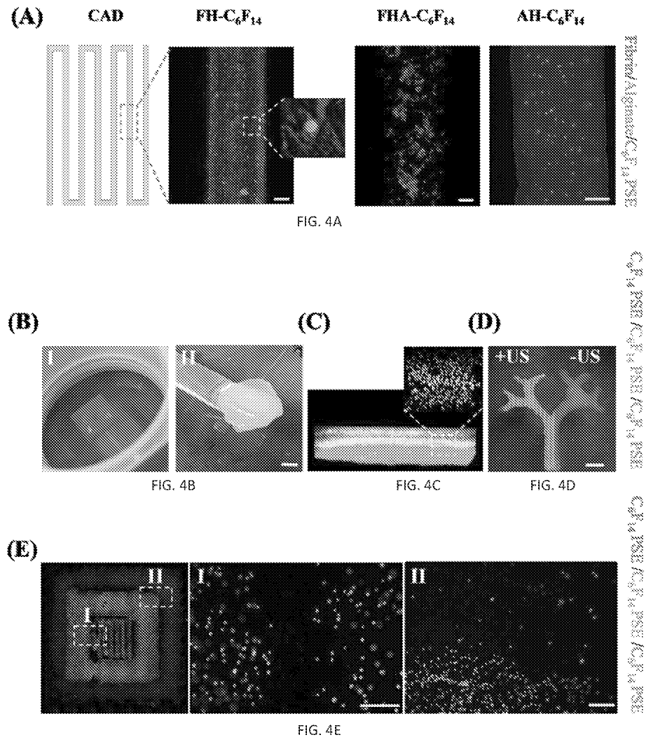

The print fidelity ratio, using the optimal printing parameters determined in the previous section, was 1.1±0.06 (n=3), 0.93±0.05 (n=3), and 1.0±0.02 (n=3) for FH-C₆F₁₄, FHA-C₆F₁₄, and AH-C₆F₁₄ bioinks, respectively (FIG. 4A). Due to slower polymerization of fibrinogen compared to alginate, FH bioink spread more. FIG. 4B displays an ARS (10 mm×10 mm×3 mm) printed with FHA-C₆F₁₄ bioink before (I) and after (II) complete polymerization. Confocal microscopy of a bioprinted bilayer ARS indicated acceptable structural fidelity (FIG. 4C). Bioprinting enabled fabrication of an ARS with a complex geometry (FIG. 4D). Fabricating customized and more complex ARSs can be attractive for biomedical applications and patient-specific therapies. Using three pneumatic printheads, an ARS with a concentric square pattern was bioprinted such that the outer (16 mm×16 mm), middle (10.6 mm×10.6 mm), and inner (5.3 mm×5.3 mm) squares contained AH-C₆F₁₄ (Ø=6.7±0.07 μm, AF₆₄₇), AH-C₆F₁₄ (Ø=6.7±0.07 μm, AF₅₅₅), and AH-C₆F₁₄ (Ø=6.7±0.07 μm, AF₄₈₈), respectively. The enlarged regions shown in FIG. 4C I &II indicate minimal bioink mixing at the boundaries. Sequential delivery of two angiogenetic growth factors from bi-layer ARSs, prepared conventionally, has been previously shown. Owing to the complexity and small size of the microvascular networks, developing reproducible, small-scale ARSs with defined patterns of release may allow for the sequential delivery of multiple payloads required to program the formation of microvasculature in vivo.

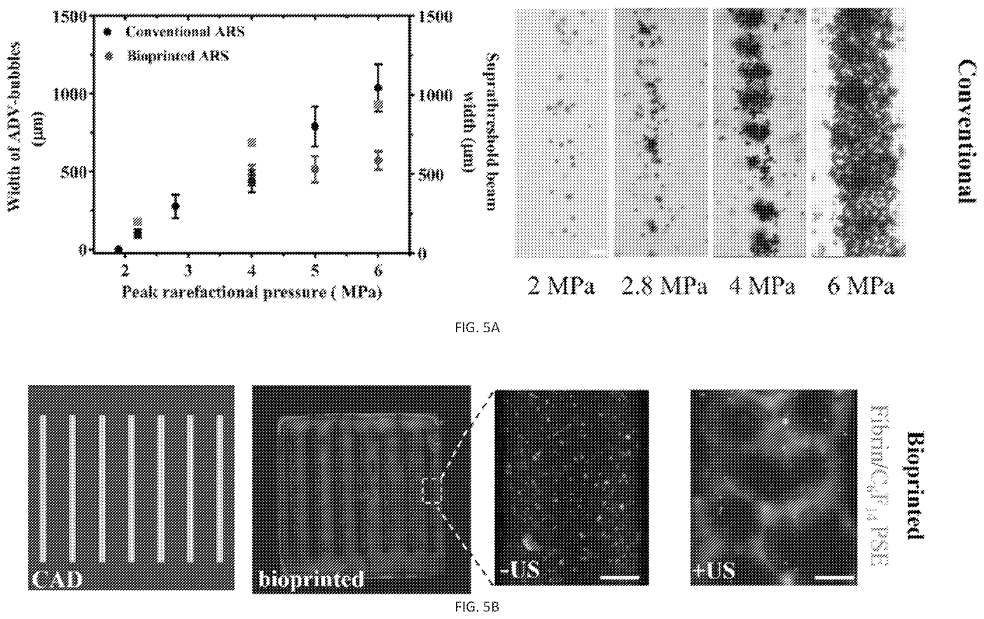

Most therapeutic applications of ultrasound utilize lower frequencies, which in turn have lower axial and lateral resolutions. Beam characterization of the transducer at the focus, using the hydrophone, yielded an axial length and lateral width at full width half maximum thickness of 3.9±0.1 mm (theoretical value: 3.28 mm) and 0.7±0.1 mm (theoretical value: 0.51 mm), respectively, at 2.5 MHz. In addition, as the peak rarefactional pressure increased, the beam width at the focus that was suprathreshold for ADV also increased (FIG. 5A, red squares), resulting in wider regions of ADV-generated bubbles in conventional fibrin ARSs (FIG. 5A, black circles). In conventional ARSs with PSEs mixed homogenously throughout, the focal width of the ADV-generated bubbles, at the peak rarefactional pressure of 6 MPa, was 1036.6±153.8 μm. At the same acoustic setting and PSE properties (e.g., size and concentration), a much smaller ADV-bubble width (572.1±58.1 μm) was generated in a micropatterned ARS containing FH-C₆F₁₄ (FIG. 5B). In extrusion-based bioprinting, the feature resolution correlates directly with the diameter of the needle. Using a 27G needle, microreservoirs with maximum printing resolution of 244.8±69.9 μm were printed using FH-C₆F₁₄ (Ø=15.6±0.07 μm, 0.05% (v/v), AF₅₅₅) and FH-C₈F₁₈ (Ø=11.9±0.09 μm, 0.5% (v/v), AF₄₈₈) bioinks (FIG. 5C).

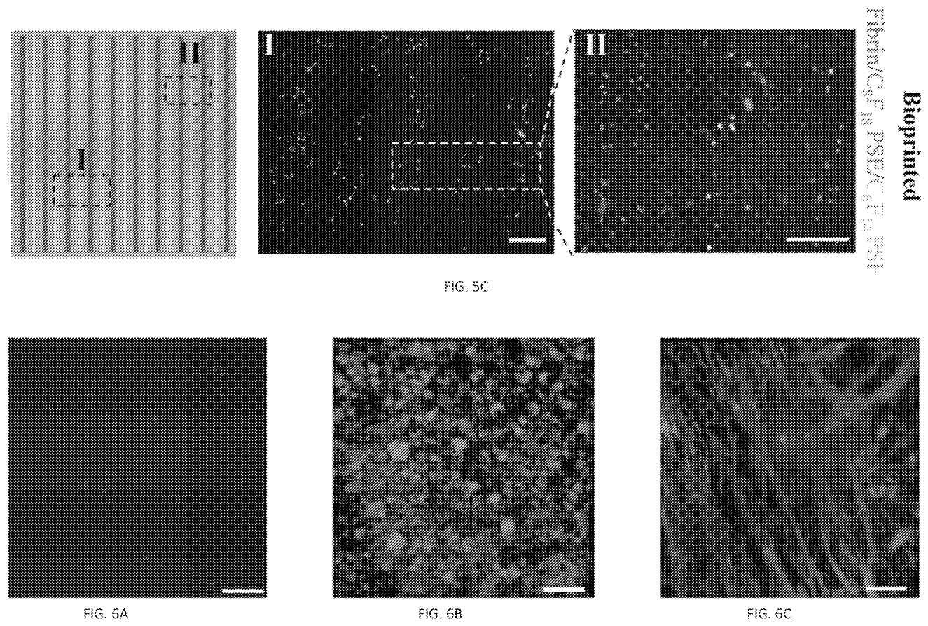

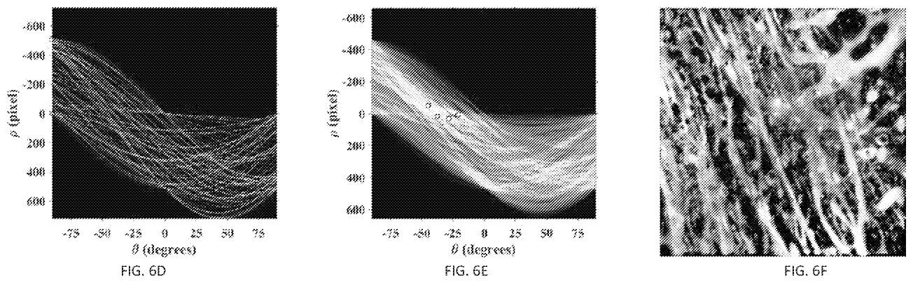

Confocal images of fibrin gels (FIG. 6A) as well as FH gels (FIG. 6B), both prepared conventionally (e.g., drop-cast), exhibited random fiber orientation. Addition of HA resulted in formation of large fibrin domains (FIG. 6B). Using similar fibrinogen and thrombin concentrations, bioprinted FH ARSs resulted in the alignment of fibrin fibers (FIG. 6C).

### Example 4

### Rheological Characterization of ARSs

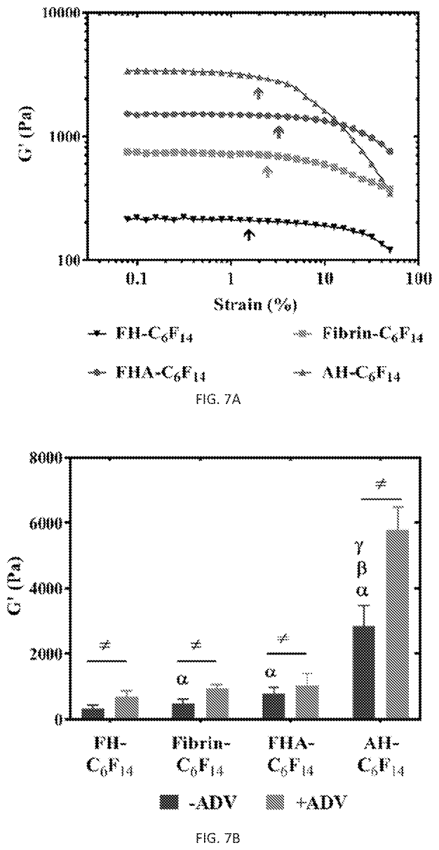

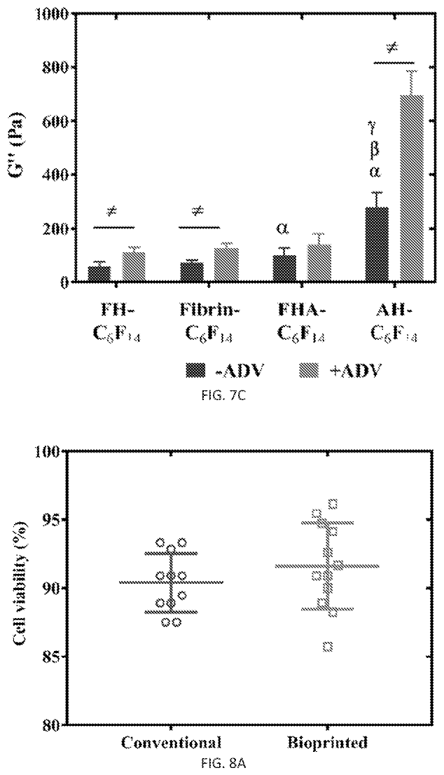

The length of LVR, determined by a strain sweep test, indicates the structural and mechanical stability of the material before the onset of structural breakdown. The critical strain was 1.5%, 2%, and 3.1% for the bioprinted ARSs made with FH-C₆F₁₄, AH-C₆F₁₄, and FHA-C₆F₁₄, respectively (FIG. 7A). Conventional fibrin-based ARSs had a critical strain of 2.5%. At the selected printing conditions, addition of alginate improved the viscoelastic moduli (FIGS. 7B & 7C). Compared to fibrin ARSs, made conventionally, printed FH ARSs exhibited a significantly lower G′ (˜1.8-fold). A previous study showed that addition of HA to fibrin gels, made conventionally, resulted in poor mechanical properties when non cross-linked HA was used. Oscillatory shear tests demonstrated that ADV caused significant increases in the bulk viscoelastic moduli of the ARSs (FIGS. 7B & 7C).

### Example 5

### Cell Studies

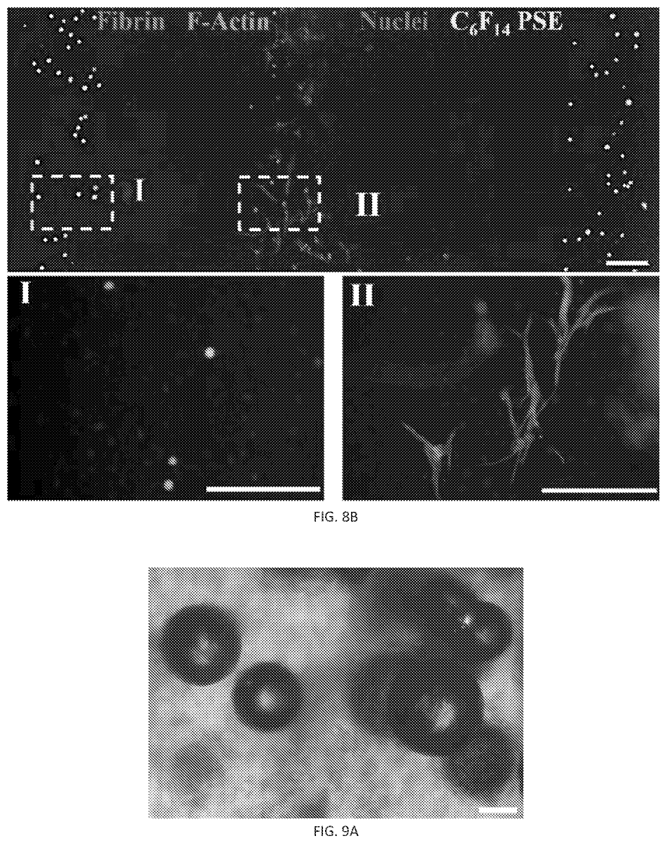

High cell viability was maintained in printed FH gels which was not significantly different than the conventionally seeded FH gels (FIG. 8A). High cell viability in fibrin-based bioprinted constructs under similar printing conditions has been shown for up to 7 days post printing. The maximum shear rate generated under the bioprinting conditions used here was 250 s-1 (FIG. 3C) which was lower than the critical shear rate of 1636 s⁻¹ reported for cell disruption. Several studies showed that shear flow alone did not cause significant cell death. The major factor contributing to cell membrane disruption was stretching caused in the extensional flow at the entrance of the syringe-needle which was significantly reduced when cells were encapsulated in shear thinning bioinks. This highlights the importance of shear thinning behavior of bioinks as a mechanism to protect cells against long exposures to shear and extensional stresses. High shear thinning characteristic in the FH bioink resulted in the generation of lower shear stress and shorter residence time, suitable for bioprinting living cells. The morphologies of bioprinted cells cultured for four days within ARSs (FIG. 8B) reveal fibroblast spread similar to those observed in fibrin-only constructs.

### Example 6

### Bioprinting-Assisted Modulation of ADV-Bubble Dynamics

The ability to control growth and collapse of the ADV-generated bubbles in ARSs could be attractive for modulating the release kinetics or migratory permissiveness of ARSs. Stable and transient bubble formation in ARSs impacted the amount as well as the rate of payload release. Payload release rate was significantly lower in ARSs with stable ADV-bubbles. Bioprinting allows precise positioning of PSEs, and in turn ADV-bubbles, in layers of different mechanical properties and therefore imposing desired bubble dynamics and release kinetics.

Extensive experimental and numerical studies on dynamic behaviors of bubble collapse near different boundaries have been reported. Interactions between a cavitating bubble and a nearby boundary can induce asymmetrical collapse and/or microjetting, where the direction of jetting can be toward (rigid boundaries) or away (soft boundaries) from the boundary, depending on mechanical properties of the boundary, bubble size, and distance from the boundary. Asymmetrical collapse and microjet formation are significantly influenced by a dimensionless standoff parameter (H), defined as the distance between the initial location of the bubble from the boundary scaled by the maximum bubble radius, and was observed for H ranging from 0.5-3.

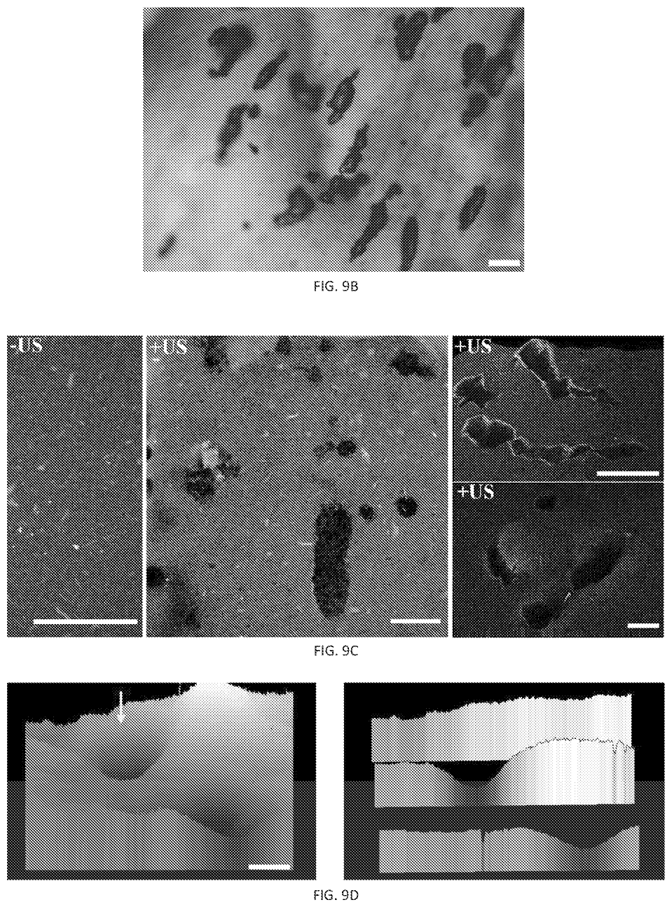

Here, to generate the condition for the ADV bubbles to undergo asymmetrical collapse and/or microjetting, a thin layer (˜200 μm) of FHA containing 0.05% (v/v) C₆F₁₄ PSE (Ø=15.6±0.07 μm) was bioprinted on a thin rigid alginate layer (˜200 μm). A maximum thickness of 200 μm was considered for the ARS to ensure that the generated ADV-bubbles would remain in the vicinity of the rigid wall (e.g., H<3). Elastic moduli of the rigid alginate and the FHA-C₆F₁₄ layers were 15.2±0.7 kPa and 0.78±0.18 kPa, respectively. Morphology of the ADV-generated bubbles were then compared in bioprinted FHA-C₆F₁₄ ARSs without (FIG. 9A) and with the presence of the rigid layer (FIG. 9B). The bilayer ARS was then imaged to identify ADV-generated features on the fluorescently-labeled rigid alginate layer. Visible pitting and indentation of the rigid alginate layer can be seen in the confocal images post-ADV (FIG. 9C). Surface profiles of confocal images indicated a maximum indentation depth of ˜35 μm in the rigid layer (FIG. 9D).

Herein, an excitation frequency of 2.5 MHz and a short pulse duration (5.4 μs) were used. Acoustic parameters such as excitation frequency, pulse duration, and the driving amplitude may impact both the dynamics and resulting morphologies of the ADV-generated features.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A composition for 3D bioprinting comprising:
0.5-5% (w/v) fibrinogen, 1-5% (w/v) alginate, or a combination thereof; and
0.1-2% (w/v) hyaluronic acid,
wherein the composition is extrudable or printable into a defined shape.

2. The composition of claim 1, wherein the composition exhibits shear thinning behavior characterized by a decreasing viscosity with increasing shear rate.

3. The composition of claim 1 or claim 2, wherein the composition has a zero-shear viscosity greater than 5 Pa·s at 20° C.

4. The composition of any of claims 1-3, further comprising 0.01-3% (v/v) of a perfluorocarbon-containing emulsion.

5. The composition of claim 4, comprising 0.01-1% (v/v) of the perfluorocarbon-containing emulsion.

6. The composition of claim 4 or 5, wherein the perfluorocarbon-containing emulsion comprises perfluorocarbon droplets which vaporize from liquid droplets into gas bubbles in response to ultrasound.

7. The composition of any of claims 4-6, wherein the perfluorocarbon-containing emulsion is a double emulsion.

8. The composition of claim 7, wherein the double emulsion is a water in perfluorocarbon in water double emulsion or an oil in perfluorocarbon in water double emulsion.

9. The composition of any of claims 4-8, wherein the perfluorocarbon-containing emulsion comprises one or more active agents.

10. The composition of claim 9, wherein the one or more active agents are conjugated to the droplet surface.

11. The composition of claim 9, wherein the one or more active agents are encapsulated within the droplet.

12. The composition of any of claims 9-11, wherein the active agent comprises a biomolecule, a therapeutic agent, a contrast agent, a detectable marker or label, or any combination thereof.

13. The composition of any of claims 1-12, further comprising a plurality of cells.

14. The composition of claim 13, wherein the cells comprise progenitor cells, undifferentiated cells differentiated cells, or a combination thereof.

15. An acoustically-responsive scaffold comprising:
a hydrogel comprising fibrin, alginate, hyaluronic acid, or a combination thereof; and
at least one spatially-patterned perfluorocarbon-containing emulsion.

16. The scaffold of claim 15, wherein the hydrogel comprises aligned fibrin fibers.

17. The scaffold of claim 15 or claim 16, further comprising an additional hydrogel layer comprising mechanical and/or rheological properties different from those of the hydrogel comprising fibrin, alginate, hyaluronic acid, or a combination thereof.

18. The scaffold of claim 17, wherein the additional hydrogel layer is a rigid hydrogel layer.

19. The scaffold of any of claims 15-18, wherein the perfluorocarbon-containing emulsion comprises perfluorocarbon droplets which vaporize from liquid droplets into gas bubbles in response to ultrasound.

20. The scaffold of any of claims 15-19, wherein the perfluorocarbon-containing emulsion comprises one or more active agents.

21. The scaffold of claim 20, wherein the one or more active agents are conjugated to the droplet surface.

22. The scaffold of claim 20, wherein the one or more active agents are encapsulated within the droplet.

23. The scaffold of any of claims 19-22, wherein the active agent comprises a biomolecule, a therapeutic agent, a contrast agent, a marker or label, or any combination thereof.

24. The scaffold of any of claims 15-23, comprising two or more spatially-patterned perfluorocarbon-containing emulsions.

25. The scaffold of any of claims 15-24, further comprising a plurality of cells.

26. The scaffold of claim 25, wherein the cells comprise progenitor cells, undifferentiated cells and/or differentiated cells.

27. A method for fabricating an acoustically-responsive scaffold comprising:
providing one or more hydrogel compositions comprising:
two or more of: 0.5-5% (w/v) fibrinogen, 1-5% (w/v) alginate, and 0.1-2% (w/v) hyaluronic acid, and
optionally, 0.01-3% (v/v) of a perfluorocarbon-containing emulsion, a plurality of cells, or a combination thereof; and
3D printing one or more layers of the one or more compositions to form an acoustically-responsive scaffold of defined shape,
wherein the acoustically-responsive scaffold comprises at least one spatially-patterned perfluorocarbon-containing emulsion.

28. The method of claim 27, comprising
providing a first hydrogel composition comprising two or more of: 0.5-5% (w/v) fibrinogen, 1-5% (w/v) alginate, and 0.1-2% (w/v) hyaluronic acid;
3D printing a first layer comprising the first hydrogel composition;
providing a second hydrogel composition comprising two or more of: 0.5-5% (w/v) fibrinogen, 1-5% (w/v) alginate, and 0.1-2% (w/v) hyaluronic acid, and 0.01-3% (v/v) of a perfluorocarbon-containing emulsion; and
3D printing a second layer comprising the second hydrogel composition,
wherein the second layer is spatially patterned in relationship to the first layer.

29. The method of claim 27 or claim 28, wherein the perfluorocarbon-containing emulsion further comprises one or more active agents.

30. The method of any of claims 27-29, further comprising providing a rigid hydrogel substrate configured to receive the one or more layers.

31. The method of any of claims 27-29, further comprising 3D printing a rigid hydrogel layer.

32. The method of any of claims 27-31, further comprising crosslinking the acoustically-responsive scaffold.

33. The method of claim 32, wherein the crosslinking comprises spraying each of the one or more layers with a crosslinking solution after 3D printing.

34. The method of claim 32 or claim 33, wherein the crosslinking comprises submerging the acoustically-responsive scaffold in a crosslinking solution.

35. The method of claim 33 or claim 34, wherein the crosslinking solution comprises thrombin and calcium chloride.

36. The method of any of claims 27-35, wherein the method results in greater alignment of fibrin fibers compared to a conventionally polymerized acoustically-responsive scaffold.

37. The method of any of claims 27-36, wherein the method results in a significantly lower storage modulus compared to a conventionally polymerized acoustically-responsive scaffold.

38. An acoustically-responsive scaffold made by a method of any of claims 27-37.

39. A method for promoting wound healing or tissue repair or regeneration, comprising implanting an acoustically-responsive scaffold of any of claims 15-26 or 38 in a desired tissue or organ in a subject.

40. The method of claim 39, wherein the tissue is a soft tissue or a hard tissue.

41. The method of claim 39 or 40, wherein the acoustically-responsive scaffold comprises non-essential amino acids, antibiotics, cytokines, growth and morphogenic factors, or a combination thereof.

42. A method for administering one or more active agents to a subject, comprising:
implanting an acoustically-responsive scaffold of any of claims 15-26 or 38 in a target site in a subject, wherein the acoustically-responsive scaffold comprises one or more active agents; and
exposing the scaffold to one or more ultrasound frequencies, acoustic pressure thresholds, or combinations thereof to deliver the one or more active agents to the target site.

43. The method of claim 42, wherein delivery of the one or more active agents is controlled spatially.

44. The method of claim 43, wherein delivery of the one or more active agents is controlled at a spatial resolution higher than dimensions of ultrasound beam.

45. The method of claim 42 or 44, wherein delivery of the one or more active agents is controlled temporally.

46. The method of any of claims 42-45, wherein any of all of the one or more active agents are delivered at the same or different times as a result of exposing the scaffold to different ultrasound frequencies, acoustic pressure thresholds, or a combination thereof.

47. Use of the acoustically-responsive scaffold of any of claims 15-26 or 38, for implants in a subject.

48. Use of the acoustically-responsive scaffold of any of claims 15-26 or 38, for administering one or more active agents to a subject.

49. Use of the acoustically-responsive scaffold of any of claims 15-26 or 38, for wound healing or tissue repair or regeneration.
